(19) United States
(12) Patent Application Publication
(10) Pub. No.: US 2024/0293519 A1
(43) Pub. Date: Sep. 5, 2024

(54) Universal mRNA Vaccine to Treat Dementia-Causing Neurodegenerative Disorders

(71) Applicants: Sarfaraz K. Niazi, Deerfield, IL (US); Matthias Magoola, Kampala (UG); Zafeer Ahmad, Wadsworth, IL (US)

(72) Inventors: Sarfaraz K. Niazi, Deerfield, IL (US); Matthias Magoola, Kampala (UG); Zafeer Ahmad, Wadsworth, IL (US)

(73) Assignee: RNA Therapeutics, Inc., Deerfield, IL (US)

(21) Appl. No.: 18/403,377

(22) Filed: Jan. 3, 2024

(51) Int. Cl. A61K 39/00

(52) U.S. Cl. CPC A61K 39/0007; A61K 2039/53; A61K 2039/70

(57) ABSTRACT

mRNA coding vaccines to treat neurodegenerative disorders (NDs), particularly Alzheimer's Disease (AD), Amyotrophic Lateral Sclerosis (ALS), Corticobasal Degeneration (CBD), Frontotemporal Dementia (FTD), Lewy Body Dementia (LBD), Multiple System Atrophy (MSA), Parkinson's Disease Dementia (PSD), and Progressive Supranuclear Palsy (PSP), to scavenge improperly folded or aggregated proteins 2MXU (PDB) (Aβ42), P10636 TAU_HUMAN (Tau Protein), P37840·SYUA_HUMAN (α-Synuclein), Q13148.TADBP_HUMAN (TDP-43), and P35637·FUS_HUMAN by generating specific antibodies based on the epitopes of these proteins. The epitopes can be linked to generate the open reading frames of mRNA vaccines.

### SEQUENCE LISTING

The instant application contains a Sequence Listing, which has been submitted electronically in ASCII format and is hereby incorporated by reference in its entirety. Said ASCII text file, created on Jan. 7, 2024, is named Universal Vaccine Sequence Listing, and is 43 kb in size.

### INTRODUCTION

Neurodegenerative diseases (NDs) caused by aggregation of proteins leading to dementia include:

Alzheimer's Disease (AD): The most common cause of dementia, characterized by amyloid-beta plaques and tau protein tangles in the brain.

Amyotrophic Lateral Sclerosis (ALS): Primarily a motor neuron disease, it can lead to dementia in some cases. It involves the aggregation of proteins like TDP-43, FUS, and SOD1.

Corticobasal Degeneration (CBD): A rare condition causing dementia, associated with abnormal tau protein accumulation in the brain.

Frontotemporal Dementia (FTD): A group of disorders causing dementia, typically involving the accumulation of tau proteins or TDP-43.

Lewy Body Dementia (LBD): A type of progressive dementia with Lewy bodies, which are abnormal aggregates of alpha-synuclein protein in neurons.

Multiple System Atrophy (MSA): Features Parkinson's-like symptoms; less common to cause dementia. Marked by glial cytoplasmic inclusions of alpha-synuclein.

Parkinson's Disease Dementia (PDD): Parkinson's disease leads to dementia in later stages, characterized by the accumulation of alpha-synuclein into Lewy bodies.

Progressive Supranuclear Palsy (PSP): Causes dementia and involves tau protein accumulation in neurofibrillary tangles, affecting different brain regions than Alzheimer's.

More specifically, the improperly folded or aggregated proteins include 2MXU (PDB) (Aβ42), P10636 TAU_HUMAN (Tau Protein), P37840· SYUA_HUMAN (α-Synuclein), Q13148.TADBP_HUMAN (TDP-43), and P35637·FUS_HUMAN. This invention presents mRNA vaccines capable of scavenging these misfolded and aggregated proteins by creating antibodies recognizing them as neoantigens, based on mRNA vaccines based on the selected epitopes of these proteins. Furthermore, the mRNA is formulated as a lipid nanoparticle that is more likely to cross the blood-brain-barrier (BBB), enabling more effective responses.

### BACKGROUND OF THE INVENTION

The Central Nervous System (CNS) constitutes the epicenter of complex neurological processes, orchestrating vital physiological functions, cognitive processes, and motor activities. A spectrum of factors contributes to the degradation of the CNS, encompassing diverse etiology ranging from genetic predispositions to environmental exposures. Neurodegenerative disorders (NDs), such as Alzheimer's Disease (AD), Amyotrophic Lateral Sclerosis (ALS), Corticobasal Degeneration (CBD), Frontotemporal Dementia (FTD), Lewy Body Dementia (LBD), Multiple System Atrophy (MSA), Parkinson's Disease Dementia (PSD), and Progressive Supranuclear Palsy (PSP) that stand as emblematic instances wherein progressive neuronal deterioration heralds cognitive decline and motor dysfunction. Beyond inherent genetic susceptibilities, external influences such as traumatic injuries, infections, and toxic exposures emerge as formidable instigators of CNS compromise.

This invention relates to preventing and treating NDs using mRNA vaccines that can generate protein-specific antibodies, both outside and across the blood-brain barrier, enabling the removal of proteins that aggregate around the nerves in the brain.

### 2MXU (PDB) (Aβ42)

The most prevalent isoforms among the Aβ peptides are Aβ40 and Aβ42, which consist of 40 and 42 residues, respectively. Amyloid beta (Aβ), particularly amyloid beta 42 (Aβ42), is a protein that can accumulate in the brain and form plaques. These plaques are made up of clumps of amyloid-beta protein. They can disrupt communication between brain cells and trigger inflammation, which can contribute to AD progression.

The pathogenesis of neurodegenerative disorders, such as AD, encompasses interconnected processes like protein aggregation, oxidative stress, neuroinflammatory response, and synaptic dysfunction. Notably, memantine is an N-methyl-D-aspartate receptor (NMDAR) antagonist; other standard therapies include rivastigmine, galantamine, and donepezil (cholinesterase inhibitors (ChEIs)). A combination therapy option involves the use of memantine and donepezil, known as Namzaric, approved for treating individuals with moderate to severe AD.

The deadliest neurodegenerative condition where Aβ amyloidogenesis appears to be the cause of Alzheimer's disease.

The examination of the structure reveals that the fibrils that make up Aβ (1-42) are disordered in residues 1-17, while residues 18-42 form a motif of β-strand-turn-β-strand that has two intermolecular, parallel, in-register β-sheets generated by residues 18-26 (β1) and 31-42 (β2). A protofilament's repeating structure can only be achieved by two molecules of Aβ (1-42). The odd-numbered residues of strand β1 of the nth molecule and the even-numbered strand β2 of the (n−1)th molecule establish intermolecular side-chain interactions. The sequence selectivity, cooperativity, and apparent unidirectional growth of Aβ fibril growth are explained by this interaction pattern, which results in partially unpaired β-strands at the fibrillar ends. It also gives fibrillization inhibitors a structural foundation.

Extracellular accumulation of Aβ plaques and the formation of neurofibrillary tangles within neurons are widely acknowledged as the leading indicators of Alzheimer's disease (AD).

The Aβ42 is more hydrophobic and mainly responsible for the aggregates formed. It can be reduced if an antibody is created specific to this antigen.

Targeting antibodies against Aβ is a viable strategy for addressing the amyloid cascade problem. Aducanumab, a monoclonal antibody that specifically targets the fibrillar form of A protein in the brain, has recently been authorized as a therapeutic intervention for AD.

### P37840·SYUA_HUMAN (α-Synuclein)

While amyloid is a general term for misfolded protein aggregates, synuclein refers to a specific protein, α-synuclein, and its isomers, involved in certain neurodegenerative diseases. They are related because both can be associated with protein aggregation in diseases, but they are not the same.

αSyn, a small protein comprised of 140 amino acids (aa), was initially identified from the electric organ synapses of the Torpedo ray. Subsequent studies revealed that αSyn is an abundant mammalian brain protein mainly expressed in presynaptic terminals and is less abundant in muscle, red blood cells, and lymphocytes. Under physiological conditions, αSyn is a natively unfolded protein structurally subdivided into three regions: an N-terminal amphipathic region (1-60 aa), a central hydrophobic nonamyloid-β component region (61-95 aa), and a highly acidic C-terminal region (96-140 aa). In addition, endogenous αSyn is also reported to form α-helical tetramers in cell cultures and brain tissue resistant to pathogenic aggregation. In the brain, αSyn is preferentially expressed in the neocortex, hippocampus, substantia nigra, thalamus, and cerebellum, where it binds presynaptic vesicles and regulates the release and reuptake of neurotransmitters, playing an important role in synaptic plasticity.

Parkinson's Disease Dementia (PDD) is a cognitive decline that can occur in the later stages of Parkinson's disease, a progressive neurological disorder primarily known for its motor symptoms like tremors, rigidity, and bradykinesia. A gradual decline in thinking and reasoning skills characterizes PDD. The exact cause of PDD is not fully understood. Still, it is believed to be related to the same processes that lead to motor symptoms in Parkinson's disease, including the loss of dopamine-producing cells in the brain, the presence of Lewy bodies, and abnormal protein deposits.

Lewy body dementia (LBD) is a complex, progressive brain disorder characterized by the abnormal deposition of proteins, known as Lewy bodies, in the brain's nerve cells. This disease is one of the most common causes of dementia after Alzheimer's disease. LBD is unique in its combination of cognitive, behavioral, and motor symptoms.

The primary protein component of Lewy bodies is α-synuclein and its isomers. This protein is found throughout the brain, but it misfolds and forms clumps in Lewy body disorders. The exact reason why these proteins aggregate and how they contribute to the symptoms of the disease is not fully understood. However, it is believed that the accumulation of these abnormal proteins disrupts normal cell function and eventually leads to cell death.

Lewy bodies affect brain areas that control movement, cognition, and behavior. This is why symptoms of Lewy body dementia can include a combination of movement disorders (like Parkinson's disease), cognitive decline, and neuropsychiatric symptoms.

Synuclein refers to a specific protein called α-synuclein and its isomers, primarily associated with NDs, including Parkinson's disease and Lewy body dementia. In these diseases, α-synuclein and its isomers aggregate in the brain, forming clumps known as Lewy bodies.

Synucleinopathies are a group of neurodegenerative diseases characterized by the abnormal accumulation of alpha-synuclein protein in neurons, nerve fibers, or glial cells. This group includes Parkinson's disease (PD), dementia with Lewy bodies (DLB), and multiple system atrophy (MSA). In PD, alpha-synuclein aggregates primarily in the substantia nigra part of the brain, leading to motor symptoms such as tremors, stiffness, and bradykinesia.

α-Synuclein and tau are abundant multifunctional brain proteins mainly expressed in neurons' presynaptic and axonal compartments, respectively. The intracellular deposition of α-synuclein or tau causes many neurodegenerative disorders, including Alzheimer's disease and Parkinson's disease. However, their exact role remains unclear, but the co-occurrence of α-synuclein and tau aggregates occurs in brain synucleinopathies and tauopathies. Furthermore, the direct interaction of α-synuclein with tau is considered to promote the fibrillization of each protein in vitro and in vivo. Both cooperate in their functional roles in the physiological conditions and pathogenesis of NDs.

Generally, the intracellular accumulation of αSyn inclusions in the brain is the major neuropathological hallmark of PD and related synucleinopathies. In contrast, the intracellular deposition of hyperphosphorylated tau aggregates is the neuropathological trait of AD and related tauopathies. Thus, major neuropathological features are quite distinct between synucleinopathies and tauopathies. Accumulating evidence suggests that αSyn contributes to the pathophysiology of AD, and tau is also known as a risk factor and mediator in the pathogenesis of PD.

### P10636 TAU_HUMAN (Tau Protein),

The tau protein is a microtubule-associated protein that plays a crucial role in stabilizing microtubules in neurons. Mutations in the tau gene are associated with various neurodegenerative diseases, including Alzheimer's disease and frontotemporal dementia.

The human tau protein is encoded by the MAPT gene, and it can undergo alternative splicing, resulting in six different isoforms. These isoforms have different numbers of amino acids and are designated as 0N, 1N, or 2N, depending on the presence of zero, one, or two inserts in the amino-terminal domain. The longest isoform, 2N4R tau, is often used as a reference in studies. It consists of 776 amino acids.

There are several isomers of tau protein: P10636-1, PNS-tau: 758; P10636-2, ON3R: 352; Tau-A: P10636-3: 316; Tau-B: P10636-4: 381; Tau-C: P10636-5: 410; Tau-D: P10636-6.

Tau is an important central nervous system protein formed by 352-441 amino acids and encoded by the MAPT (microtubule-associated protein tau) gene on chromosome 17, which generates 6 isoforms. Tau is in axons, dendrites, nuclei, cell membranes, and synapses of neurons. The protein is also expressed to a lesser extent in astrocytes and oligodendrocytes, although its role in these cells has been little investigated. The protein is also present in the interstitial fluid and can cross into the cerebrospinal fluid (CSF) and reach the systemic circulation. The primary function of tau is to promote the assembly and stabilization of microtubules in neuronal axons. Tau also plays a role in various other biological processes, including myelination, neurogenesis, motor function, learning, and memory. Tau binding to microtubules is regulated by its phosphorylation/dephosphorylation equilibrium. In physiological conditions, tau is unfolded and phosphorylated.

At the same time, the pathological form is characterized by an excess of hyperphosphorylation, leading to disengagement from the microtubules and conformational changes that lead to the formation of paired helical and straight filaments of abnormally phosphorylated tau and subsequently to tau aggregates. These aggregates can cause degeneration of neurons and glial cells, leading to various clinical cognitive, behavioral, and motor manifestations, which are classified into neurodegenerative disorders called ‘tauopathies.’ Tauopathies are classified into primary and secondary tauopathies. In primary tauopathies, the abnormal tau accounts for the underlying neurodegenerative process. Primary tauopathies include progressive supranuclear palsy (PSP), corticobasal degeneration, corticobasal syndrome tauopathy, FTD, frontotemporal dementia, frontotemporal lobar degeneration, primary progressive aphasia, MAPT mutation, argyrophilic grain disease, and primary age-related tauopathy. In secondary tauopathies, tau neuronal inclusions are associated with the extracellular deposition of a second aggregated protein. Secondary tauopathies include Alzheimer's disease (AD) and Down syndrome (in which Aβ accumulates), Lewy body dementia (in which α-synuclein accumulates), and chronic traumatic encephalopathy (in which TAR DNA-binding protein 43) accumulates.

Recently, it has been shown that tau is differentially phosphorylated in various tauopathies. In the brain of AD patients, there is increased phosphorylation at positions Ser202, Thr231, and Ser235, while FTD brains show increased phospho-Ser202 and argyrophilic grain dementia brains show increased phospho-Ser396. In neurodegenerative tauopathies, pathological tau can propagate between neuroanatomically connected brain regions by multiple mechanisms, spreading tau pathology throughout the brain. However, recent neuropathological studies in the AD brain suggest that local replication, rather than spreading between brain regions, is the main process driving the overall rate of tau accumulation in neocortical regions. There are also contrasting theories on whether soluble or aggregated tau species correlate with disease progression and cognitive decline in AD patients.

The conversion of soluble αSyn and tau into insoluble amyloid-like fibrils and aggregates is the central event in the development of neurodegeneration. Mutations in the SNCA or MAPT gene promote protein aggregation and disease progression. Interestingly, distinct αSyn strains displayed different efficiencies in cross-seeding tau aggregation in neuronal cells and in human P301S tau transgenic mice.

Tauopathies are a group of neurodegenerative diseases marked by the abnormal accumulation of tau protein in the brain, leading to various cognitive and motor impairments. Among these, Alzheimer's Disease is the most prevalent, characterized by both tau tangles and amyloid plaques, primarily affecting memory and cognitive function. Progressive Supranuclear Palsy (PSP) and Corticobasal Degeneration (CBD) are rarer forms, impacting movement, balance, and coordination due to tau accumulation in specific brain regions like the basal ganglia and brainstem. Frontotemporal Dementia (FTD) represents a spectrum of disorders causing progressive degeneration in the brain's frontal or temporal lobes, with tau tangles being a common pathological feature in some subtypes. Chronic Traumatic Encephalopathy (CTE), often associated with repeated head trauma, exhibits widespread tau deposits across the brain. Additionally, FTD, a rare dementia, involves the progressive loss of nerve cells and tau tangle formation. While the exact mechanisms of tauopathies remain unclear, current treatments mainly focus on symptom management, with ongoing research aimed at unraveling their complexities and developing effective therapies.

### Q13148.TADBP_HUMAN (TDP-43)

The transactive response (TAR) DNA-binding protein 43 (TDP-43) is another protein commonly found in FTD cases, especially in those with the behavioral variant and some forms of primary progressive aphasia. TDP-43 pathology is involved in a range of neurological disorders, including some cases of ALS (amyotrophic lateral sclerosis).

Frontotemporal dementia (FTD) is a cluster of brain disorders that primarily affect the frontal and temporal lobes, where the identification of aggregating proteins is crucial for understanding its pathology. Unlike Alzheimer's disease, which is marked by the accumulation of amyloid-beta plaques and tau protein tangles, FTD involves different proteins. In some cases of FTD, the tau protein accumulates abnormally in the brain. This is similar to Alzheimer's, but the pattern of deposition differs. Tau-positive FTD is often linked with certain genetic mutations.

### P35637·FUS_HUMAN

In a smaller percentage of cases, the FUS protein aggregates in neurons. FUS pathology is less common than tau or TDP-43 in FTD but is significant in understanding the disease's mechanisms.

The accumulation and aggregation of these proteins are thought to disrupt normal cellular functions, leading to the death of nerve cells in the frontal and temporal lobes of the brain. This cell death is what causes the symptoms of FTD. The exact reason why these proteins accumulate abnormally is still under investigation, but genetic factors and cellular processes like protein folding, processing, and degradation play a role.

### Blood Brain Barrier

A significant obstacle to effective medication administration to the central nervous system (CNS) has been the BBB. Because of this, most brain disorders are incurable. Because large molecules usually have low BBB permeability, it is often discouraged to develop large-molecule medicines. Many potential large-molecule modern drugs, otherwise effective in ex-vivo studies, have not been developed for clinical use because they cannot be delivered into the CNS) in sufficient quantities. These drugs include engineered proteins (e.g., nerve growth factors), antibodies, genes, vectors, micro-RNA, siRNA, oligonucleotides, and ribozymes.

Tight junctions, created by the interaction of several transmembrane proteins that protrude into and seal the paracellular pathways, form the blood-brain barrier (BBB). The intricate interplay between these junctional proteins-occludin and claudin-effectively prevents polar solutes from blood from diffusing freely along these possible paracellular channels in water, preventing them from entering the cerebrospinal fluid. The following techniques to cross the BBB have been developed due to significant scientific work. The use of liposomes or other charged lipid formulations, which have limited complex stability in serum and high toxicity over time. Electroporation-based techniques are only effective when carried out in healthy cells during a specific window of development, leading to a loss of bioactivity or expression, and viral-based vectors and fusions which have raised several safety concerns and have only limited efficacy in humans and animals. Typically, these methods require invasive procedures like direct injection into the brain to achieve targeted delivery.

Antibodies can uniquely target specific receptors on the endothelial cells lining the BBB, enabling receptor-mediated transcytosis. This process involves binding antibodies to these receptors, initiating an internalization process that transports them and their attached drug cargo across the BBB. The efficacy of antibodies in drug delivery can be enhanced by reducing their affinity for a transcytosis target, consequently boosting their brain uptake.

Nanobodies, a novel class of antibody fragments, are gaining significant attention in biomedical research due to their unique properties. Originating from camelid antibodies, nanobodies consist of a single variable domain of heavy-chain antibodies (VHH) and are the smallest known naturally occurring intact antigen-binding fragments. Their small size, high stability, and solubility make them highly desirable for various therapeutic and diagnostic applications, including AD.

One way to cross the BBB is using a nano lipid formulation comprising the steps of mixing D-Lin-MC3-DMA, DSPC, cholesterol, and DMG-PEG 2000 in an absolute ethanol solution, adding the mixture into a citrate buffer solution, and extruding the mixture by a liposome extruder to obtain the liposome nanoparticle (LNP). Current mRNA vaccines use LNP formulation that can help cross the BBB and, in many ways, serves as an adjuvant to mRNA vaccines.

mRNA Vaccines

The heart of the mRNA is the Coding Sequence, comprising codons, which are nucleotide triplets that dictate the amino acid sequence in the resulting protein. Following the coding sequence is the 3′ Untranslated Region (3′ UTR), which, like its 5′ counterpart, doesn't code for protein but plays a role in mRNA stability and translation regulation. Finally, the Poly-A Tail, a string of adenine nucleotides at the mRNA's 3′ end, further stabilizes the mRNA and influences its lifespan for translation, assisting in its transport from the nucleus to the cytoplasm. Additionally, Ribosome Binding Sites, primarily located within the 5′ UTR, are critical for correct ribosome assembly and translation initiation on the mRNA.

The number of protein molecules generated from a single mRNA is primarily determined by “translation efficiency.” The stability of the mRNA molecule, the availability of different translation components, and the existence of translation initiation sites are some factors affecting translation efficiency.

The length of the mRNA, translation efficiency, and stability of the resultant protein all affect how many protein molecules can be translated from a single mRNA molecule. It is noteworthy that translation is a dynamic process and that a chain of ribosomes known as polysomes can be formed when multiple ribosomes simultaneously translate the same mRNA molecule. Various ribosomes can translate a single mRNA molecule; this phenomenon is known as polysome or ribosome “clustering.” This makes it possible to produce several protein molecules from the same mRNA template effectively and concurrently. Several factors, including ribosome availability, cellular circumstances, and particular mRNAs and their associated regulatory elements, govern how many ribosomes can translate mRNA simultaneously.
Step 1: Select an Antigenic Epitope: Start by identifying a specific sequence or epitope from a protein that is known to be antigenic. Antigenic epitopes are protein regions the immune system recognizes, typically as part of an antibody-antigen interaction. In the case of AD, both types yield the same epitope, so the latter modification is the most important.
Step 2: Design mRNA Sequence: Design an mRNA sequence that encodes the selected epitope. The mRNA sequence should follow the rules of mRNA transcription, such as starting with a 5′ cap and including a 3′ poly-A tail. Ensure the sequence is in-frame with the ribosome so that translation produces the desired epitope.
Step 3: Codon Optimization: Optimize the mRNA sequence for translation efficiency in the desired host cell. This may involve choosing more frequently used codons in the host organism to ensure efficient translation.
Step 4: Consider mRNA Modifications: To enhance stability and translation efficiency, consider incorporating modified nucleotides, such as pseudouridine or 5-methylcytidine, into the mRNA sequence. These modifications can improve mRNA stability and reduce immune recognition. Also significant is the replacement of uridine with pseudouridine.
Step 5: Delivery Method: Determine how to deliver the mRNA to the target cells. This can include electroporation, lipid nanoparticles, or viral vectors.
Step 6: Expression System: Choose an appropriate expression system for mRNA, such as a cell line or organism that can efficiently translate the mRNA and produce the epitope.
Step 7: In Vitro Translation: Transcribe and translate the mRNA in an in vitro translation system, such as a cell-free translation system or using cultured cells. This will help verify that the mRNA is producing the desired epitope.
Step 8: Antigen Presentation: Once the mRNA has been translated into the antigenic peptide within the target cells, it can be processed and presented on the cell surface by major histocompatibility complex (MHC) molecules. This presentation is essential for immune recognition.
Step 9: Immunization: Use the translated peptide to immunize animals or individuals to stimulate an immune response. Use this antigenic peptide as part of a vaccine or immunotherapy.
Step 10: Immune Response Evaluation: Monitor the immune response by measuring the production of antibodies or T-cell responses against the antigenic peptide. Use techniques such as ELISA, flow cytometry, or cytokine assays to assess the immune response.

The complex process of matching a sequence of the Fc region of an antibody to an epitope sequence involves a synergy of bioinformatics and molecular biology techniques. Discerning the distinct roles and structures of the Fc region and epitope is pivotal. The Fc region interacts with cell surface receptors and complement proteins at an epitope, the specific portion of an antigen recognized by the immune system.

The second phase entails retrieving and aligning these sequences, a process facilitated by tools and databases like GenBank and BLAST. When 3D structures are obscure, homology modeling tools become instrumental in predicting these structures. Software such as PyMOL and UCSF Chimera enables researchers to visualize and analyze these structures in detail.

The enigma of the interaction between the Fc region and the epitope begins to unravel during the docking studies. Tools like HADDOCK and ClusPro simulate these intricate interactions, revealing binding affinities and interactive sites. Experimental validation, however, is indispensable. Techniques like site-directed mutagenesis and binding assays, such as ELISA or Surface Plasmon Resonance, are deployed, providing empirical data that corroborate the in-silico findings.

Data interpretation is another crucible where statistical tools play a significant role. They sift through the conglomerate of data, delineating substantial patterns and insights and leading to coherent conclusions. Further in vivo studies augment these findings, offering a comprehensive view of the Fc-epitope interactions within a biological context.

The B epitope linkers are short peptide sequences that connect different B cell epitopes to create chimeric proteins or multi-epitope antigens for various applications, including vaccine development. NEURODEGENERATIVE DISORDER VACCINES

mRNA vaccines utilize in vivo ribosomes to express or translate antigens that can create antibodies. To treat NDs, antibodies against the α-synuclein, its isoforms, and tau proteins can be created by mRNA molecules capable of coding the epitopes of these proteins; creating the entire protein will serve a negative purpose, as it will increase the concentration of these proteins. Besides, the humoral system is already exposed to these proteins and has been unable or inaccessible to this protein, gone rogue.

Creating antibodies against an antigen like the α-synuclein and its isomers or tau protein can be achieved by creating a coding mRNA based on the epitopes of these proteins, particularly the B-cell epitopes.

An epitope, also known as an antigenic determinant, is a specific part of an antigen recognized and bound by an antibody, B-cell receptor, or T-cell receptor during an immune response. Antigens are substances (often proteins) that can trigger an immune response in the body, and they may be part of a pathogen such as a virus or bacterium or a foreign substance like pollen. Epitopes can be categorized into two main types. The linear or sequential epitopes consist of a linear sequence of amino acids within the antigen's primary structure. They are recognized by their amino acid sequence rather than their three-dimensional structure. The conformational or discontinuous epitopes are formed by amino acids that are not sequential in the primary protein sequence but are brought together in space by protein folding. These epitopes are recognized by their three-dimensional structure. Given any amino acid sequence, the B and T-cell epitopes can be calculated (http://tools.iedb.org).

The interaction between an epitope and an antibody or a receptor is specific; a particular epitope will bind to a specific antibody or receptor with high specificity. This specificity is fundamental to the immune system's ability to detect and respond to various antigens. Understanding epitopes is crucial in multiple fields, including vaccine development, immunotherapy, and diagnostic testing.

Epitopes are the specific regions of antigens (in this case, the autoantibodies) that are recognized by autoantigens. For MHC binding, epitopes must bind to major histocompatibility complex (MHC) molecules to be presented to T cells. For class I MHC, epitopes are typically 8-11 amino acids in length, while for class II MHC, they are usually longer, around 15-24 amino acids. Some epitopes may be discontinuous and composed of amino acids not adjacent to the protein sequence. B cell epitopes are usually 5-17 amino acids in length but can be extended without adverse effects on the immune function.

B cell epitopes are typically fragments located on the outer surface of a (native) protein or peptide antigens, preferably having 8 to 15 amino acids, which may be recognized by antibodies, i.e., in their native form. This invention used a 5-25 amino acid sequence cutoff since epitopes are rarely found beyond these limits. Such epitopes of proteins or peptides may be selected from any of the variants of such proteins or peptides. In this context, antigenic determinants can be conformational or discontinuous epitopes, which are composed of segments of the proteins or peptides as defined herein that are discontinuous in the amino acid sequence of the proteins or peptides as defined herein but are brought together in the three-dimensional structure or continuous or linear epitopes which are composed of a single polypeptide chain. In the context of the present invention, an epitope may be the product of the translation of a provided coding mRNA as specified herein.

The next step is to convert the epitope sequences into nucleoside coding sequences by first converting the target polypeptide sequence into DNA through reverse transcription and then to RNA. Multiple epitopes from the same protein or from different proteins can be linked together using linkers including but not limited to: AAAGY (Alanine-Alanine-Alanine-Glycine-Tyrosine), AAY (alanine and tyrosine), APAAP (Alanine-Proline-Alanine-Alanine-Proline), EAAAK (Glutamic Acid-Alanine Linker), EFGGG (Glutamic Acid-Phenylalanine-Glycine-Glycine-Glycine), GGAGG (A slight variation of the GGGGS linker with an alanine residue), GGGGS (Glycine-Serine Linker), GGGS (linker is one of the simplest and commonly used linkers), GGGSGGG (linker consists of a longer sequence of glycine (G) and serine (S) residues), GGGSGGGGSGGG (linker with multiple glycine and serine residues), GGPGG (Glycine-Glycine-Proline-Glycine-Glycine), GGSGG: (An inversion of the standard GGGGS linker), GGSSG (Glycine-Glycine-Serine-Serine-Glycine), GGTGG (Glycine-Glycine-Threonine-Glycine-Glycine), GPGP (Glycine-Proline-Glycine-Proline), GPGPG (Glycine-Proline-Glycine-Proline-Glycine), GPGS (Glycine-Proline-Glycine-Serine), GSGPG (Glycine-Serine-Glycine-Proline-Glycine), GSSG (Glycine-Serine-Serine-Glycine), GSSGG (Glycine-Serine-Serine-Glycine-Glycine), GSTSG (Glycine-Serine-Threonine Linker), KK (Lysine-Lysine), KKKGS (Lysine-Glycine-Serine Linker), KLPGWSG: (A specific sequence), LEGGGS (Leucine-Glutamic Acid-Glycine-Glycine-Serine), NPGP (Asparagine-Proline-Glycine-Proline), SGGGG: (A variant of the GGGGS linker), SGSGS (Serine-Glycine-Serine-Glycine-Serine), SSGGG (Serine-Serine-Glycine-Glycine-Glycine), SSGSS (Serine-Serine-Glycine-Serine-Serine), TGGGS (Threonine-Glycine-Glycine-Glycine-Serine), TPGTG (Threonine-Proline-Glycine-Threonine-Glycine), TPP (Proline-Proline-Threonine), TPTPPT (Threonine-Proline-Threonine-Proline-Proline-Threonine), TSGSG: (A variant of the GSTSG linker), , TSGTSG (Threonine-Serine-Glycine-Threonine-Serine-Glycine), and XTEN: (A synthetic, non-immunogenic linker).

mRNA vaccine functional regulation requires untranslated regions (UTRs) between the open reading frame (ORF) and the 5′ and 3′ ends, upstream and the downstream of the mRNA. These UTRs contain regulatory sequences associated with mRNA stability and efficient and correct mRNA translation. They also help recognize mRNA by ribosomes and help in post-transcriptional modification of the mRNA. The mRNA translation and its half-life can be improved by including cis-regulatory sequences in the UTRs. Additionally, the inclusion of naturally occurring sequences, such as those derived from alpha- and beta-globins, have been widely used to design mRNA constructs for vaccines.

The in vitro transcribed (IVT) mRNA has a polyadenylated section at its 3′ end known as the poly(A) tail that is essential for determining the mRNA's lifespan. The poly(A) tails of the naturally occurring mRNA molecules in mammalian cells have a longer length of approximately 250 nucleotides (nt), gradually shortened throughout the lifespan of mRNA in the cytosol. Since the tail size affects mRNA degradation, incorporating poly(A) tails is desirable in producing mRNA with a longer half-life. The addition of approximately 50-250 nt to the poly(A) tail is optimal to produce mRNA with the desired prolongation of degradation.

mRNA Vaccines Manufacturing

mRNA vaccine production can be divided into three phases: upstream mRNA manufacturing, downstream mRNA purification, and formulation of mRNA lipid nanoparticles. mRNA production can be performed in a one-step co-transcriptional reaction, where a capping reagent is used, or in a two-step reaction, where the enzymatic capping is performed. mRNA purification process at a smaller lab-scale consists of DNase I digestion enzyme followed by LiCl precipitation of the mRNA. Purifying mRNA at a large scale involves utilizing well-established chromatographic methods coupled with tangential flow filtration (TFF). Finally, the formulation of mRNA vaccines consists of mixing an aqueous mRNA solution with a lipid solution in a non-aqueous phase. This causes the self-assembly of the lipid nanoparticles (LNPs) and encapsulates the negatively charged mRNA within the core of the LNPs. Mixing the mRNA and the lipid molecules in a staggered herringbone micromixer (SHM) occurs in various cycles, forming the final mRNA-LNP vaccines.

### Upstream Production

The upstream production of mRNA vaccines comprises the generation of the mRNA transcript from the plasmid containing the gene of interest. This reaction is called the in vitro transcription reaction (IVT). The IVT enzymatic reaction relies on RNA polymerase enzymes such as T7, SP6, or T3. The RNA polymerase enzymes catalyze the synthesis of the target mRNA from the linearized DNA template containing the gene of interest. A linearized DNA template is produced by the cleavage of a plasmid containing the gene of interest by restriction of endonuclease enzymes, or amplification of the gene of interest by PCR can also produce mRNA molecules.

The essential enzymes of an IVT reaction include (i) RNA polymerase—which converts DNA to RNA; (ii) inorganic pyrophosphatase (IPP)—increases IVT reaction yield; (iii) guanylyl transferase-which adds GMP nucleoside to 5′ ends of mRNA, (iv) Cap 2′-O— Methyltransferase (SAM)—this enzyme adds a methyl group at the 2′ positions of the 5′ cap of the mRNA, (v) DNase I—endonuclease used for removal of contaminating genomic DNA from RNA samples and degradation of DNA templates in the IVT reaction, and (vi) poly(A) tail polymerase and (vii) modified and unmodified nucleoside triphosphates (NTPs). These enzymes facilitate the upstream development of the mRNA transcript from a plasmid containing the gene of interest.

The capping enzymes include SAM and guanylyl transferase, which enzymatically form a 5′ cap at the 5′ end of the mRNA. In contrast, the poly(A) tail polymerase tailing enzyme forms the poly(A) tail. Another method of 5′ capping uses the co-transcriptional method, where the 5′ cap is prepared previously, and this cap is added to the mRNA non-enzymatic. This co-transcription reaction can be performed using CleanCap® Reagent AG.

### Downstream Purification

mRNA is produced by the IVT reaction in the upstream production phase; it is then isolated and purified by multiple purification steps in downstream processing. The IVT reaction mixture contains several impurities, including residual NTPs, enzymes, incorrectly formed mRNAs, and DNA plasmid templates. Lab-scale purification of IVT mRNA involves methods based on DNA removal by DNase enzyme digestion followed by lithium chloride (LiCl) precipitation.

The lab-based methods do not allow the complete removal of aberrant mRNA species, including dsRNA and truncated RNA fragments. Removing these impurities is essential and critical to obtaining a pure mRNA product that demonstrates its intended efficacy and safety profile. Yields 10-1000-fold can increase mRNA transfection and related protein production if reverse-phase HPLC purifies modified mRNA before its delivery to dendritic cells.

Chromatography is a commonly and widely used purification process accepted in the biopharmaceutical industry for purifying vaccines and biological drug products. The first published procedure in 2004 for large-scale nucleic acid purification of RNA oligonucleotides used size exclusion chromatography (SEC). SEC has several advantages, including selectivity, scalability, versatility, cost-effectiveness, and high purity and yields for nucleic acid products.

However, SEC cannot remove the same size impurities, such as dsDNA. Instead of SEC, ion-pair reverse-phase chromatography (IEC) is an excellent purification technique for mRNA vaccines. IEC can easily separate the target mRNA from the IVT reaction impurities. This separation method relies on the charge difference between the target mRNA and the impurities.

IEC has several advantages, including separating longer RNA transcripts from the target mRNA, higher binding capacity, cost-effectiveness, and scalability. The process becomes complex and temperature-sensitive since IEC is performed under denaturing conditions [84]. Affinity-based chromatographic separation is another mRNA purification method. Deoxythymidine (dT)-Oligo dT is a sequence that captures the mRNA's poly(A) tail. Chromatographic beads containing Oligo dT can be used for the downstream purification of mRNA vaccines.

Tangential flow filtration (TFF) or core bead filtration can remove small-sized impurities. As a final polishing step for mRNA vaccines, hydrophobic interaction chromatography (HIC) connected to a connective interaction media monolith (CIM) column containing OH or SO3 ligands can be extremely beneficial.

### Formulation

mRNA molecules, being negatively charged, should be formulated in a lipid-based drug delivery system to avoid mRNA degradation and improve its transfection efficiency and half-life. LNPs are the most trustworthy, reliable, and US FDA-approved lipid-based non-viral carrier system for delivering mRNA vaccine drug substances. mRNA LNPs are formed by precipitating lipids dissolved in an organic phase and mixing them with mRNA in an aqueous phase. The most used lipids in the organic phase are ionizable, cholesterol, helper lipids, and PEG-lipids.

Meanwhile, the mRNA is dissolved in a citrate or acetate buffer at pH 4. Mixing the aqueous and non-aqueous solutions protonates the ionizable lipid, causing an electrostatic attraction between the ionizable protonated lipid and the anionic mRNA. This interaction is simultaneously coupled with the hydrophobic interactions of other lipids. It drives spontaneous self-assembly of the mRNA-LNPs with the mRNA encapsulated within the core of the nanoparticles. This process is also called microprecipitation. Following LNP formation, they are dialyzed to remove the non-aqueous solvent, usually ethanol, and the solution pH is elevated to physiological pH.

Microfluidic mixers enable the formation of small-sized LNPs with a low polydispersity index and high mRNA encapsulation efficiency. Microfluidic mixing is the most used method for mRNA LNP formulation at the lab scale and GMP level. The Precision NanoSystems' NanoAssemblr® platform has been widely used for LNP formulation development and GMP production under controlled environments. This system uses a staggered herringbone micromixer (SHM) cartridge architecture. The structure of SHMs enables the two aqueous and non-aqueous solvents to mix within microseconds. This timescale is much smaller than the time required for lipid aggregation; hence, SHMs produce small nanoparticles of uniform size.

The NanoAssemblr® settings can be simply adjusted to change the aqueous and non-aqueous phase's flow rate and volume to obtain LNPs of the desired size and size distribution. A total flow rate of 12-14 mL/min and a flow rate volume ratio of 3:1, non-aqueous: aqueous phase, is commonly used to generate small monodisperse LNPs. Although SHMs have several advantages for the efficient production of LNPs, their utility in GMP manufacturing is limited due to solvent incompatibility. The long-term exposure of the SMH and its internal parts containing polydimethylsiloxane to ethanol can lead to its deformation. It becomes difficult to replace the cartridges in a continuous GMP manufacturing run. Hence, T-mixers are utilized for LNP scale-up and manufacturing. They can produce LNPs like the SMH, handle higher flow rates and volumes (60-80 mL/min), and are compatible with organic solvents such as ethanol.

mRNA Delivery

mRNA vaccine molecules are large (104-106 Da) and negatively charged. They are unable to pass through the lipid bilayer of cell membranes. Naked mRNA would be destroyed and degraded by the nucleases in the bloodstream. In addition, naked mRNA is also attached and engulfed by immune cells in the tissue and the serum. Methods to deliver mRNA molecules into the cells include gene guns, electroporation, and ex vivo transfection. The in vivo methods of delivering mRNA involve transfection immune or non-immune cells using lipids or transfecting agents.

Although naked mRNA, liposomes, and polyplexes have shown clinical effectiveness in humans, LNPs for mRNA vaccines are the only drug delivery system that has demonstrated clinical efficacy and has been approved for human use. The COVID-19 mRNA vaccines against SARS-COV-2, developed by Moderna and Pfizer/BioNTech, employ LNPs to deliver the mRNA payload to the body. LNPs are currently the foremost non-viral delivery vector employed for gene therapy. The clinical effectiveness of LNPs was first demonstrated when LNP-siRNA therapeutic Onpattro® (patisiran) was approved by the US FDA for hereditary transthyretin-mediated amyloidosis. LNP formulations are the most successful, effective, and safe method of delivery of mRNA vaccines for human immunizations. LNPs offer numerous advantages for mRNA delivery to the site of action, including ease of formulation and scale-up, highly efficient transfection capacity, low toxicity profile, modularity, compactivity with different nucleic acid types and sizes, protection of mRNA from internal degradation, and increasing the half-life of mRNA vaccines. LNPs are typically composed of four components: an ionizable cationic lipid, a helper phospholipid, cholesterol, and a PEGylated lipid. These lipids encapsulate the mRNA vaccine's payload and protect the nucleic acid core from degradation.

### Cationic and Ionizable Lipids

Cationic lipids were the first generation developed and utilized for mRNA vaccine delivery. These lipids contain a quaternary nitrogen atom, imparting them a permanently positive charge. The positive charge of these lipids enables them to form ionic interactions with the negatively charged mRNA vaccines, forming a lipid complex called a lipoplex. DOTMA and its synthetic analog DOTAP were the first cationic lipids to deliver mRNA vaccines. Cationic lipids such as DOTMA, DOPE, and DOGS have been widely used for mRNA delivery since then, including the commercially available and successful Lipofectin, a mixture of DOPE and DOTMA, and is one of the first LNP formulations, proving successful in vivo translation of mRNA.

The early cationic lipids demonstrated promising gene delivery in vitro but suffered from inadequate in vivo efficacy. The positive charge of the nitrogen head group and the non-biodegradable nature of the early cationic lipids were responsible for their ineffective delivery and efficacy in vitro [40]. Ionizable lipids, also called pH-dependent ionic lipids, are the second generation of cationic lipids containing a primary amine, which imparts them a positive charge at or below physiological pH. The property of these lipids, having a neutral charge in the bloodstream at physiological pH, helps improve their safety compared to first-generation cationic lipids. They also extend the circulation time of the LNPs as compared to LNPs derived from cationic lipids. These were developed to overcome the shortcomings and safety issues, such as immune activation and interaction with serum proteins of the first-generation cationic lipids. DLin-MC3-DMA was the first US FDA-approved ionic lipid used in the first siRNA drug, Onpattro®. The DLin-MC3-DMA ionic lipid was synthesized after a series of modifications on the first ionic lipid, DODMA. DLinDMA was formed by replacing the oleyl tails of DODMA [42,43]. DLinDMA demonstrated superior ability to DODMA in protective immunity against the respiratory syncytial virus (RSV) in vivo. DLinDMA is further optimized to DLin-KC2-DMA and DLin-MC3-DMA depending on a series of structure-activity relationship-based studies. DLin-MC3-DMA is considered the first generation of ionizable lipids.

Including ester moieties helped increase the biodegradability of MC3 and systemic clearance. Ester moieties are easy to install in a lipid, biodegradable, chemically stable, and easily cleaved by the intracellular esterase. MC3 was an essential precursor and a starting point for developing biodegradable ester ionizable lipids. Ester-based biodegradable ionizable lipids have demonstrated higher potency in gene delivery than MC3.

The third-generation ionizable lipids are optimized, having a limited number of chemical synthesis steps, which increases the high-throughput production of the ionizable lipids; 98N12-5 is the first example of a third-generation ionizable lipid.

### PEG-Lipid

Among the ingredients, polyethylene glycol (PEG) is a hydrophilic material well known for various cosmetic, food, and pharmaceutical applications. The PEGylated lipid component in LNPs is usually linked to an anchoring lipid. PEG was found to be an essential chemical in the formulation of LNPs to mitigate the uptake of nanoparticles by filter organs, also improving the colloidal stability of LNPs in biological fluids. Hence, circulation half-life and in vivo distribution of LNPs are enhanced.

Usually, PEG-lipids account for a minimal molar % among lipid constituents in LNPs (approximately 1.5%). However, they are pivotal in affecting crucial parameters such as population size, polydispersity index, aggregation reduction, particle stability improvement, and encapsulation efficiency. The molecular weight of PEG and the carbon chain length of the anchor lipid can be exploited to fine-tune the time of circulation and uptake by immune cells, altering the efficiency.

Additionally, the PEG-lipid coat on LNPs acts as a steric hydrophilic barrier for preventing self-assembly and aggregation during storage. Therefore, the presence of PEG helps stabilize the LNP and regulates size by limiting lipid fusion. The amount of PEG is inversely proportional to the size of the LNP; the higher the PEG content, the smaller the size of the LNP. Generally, the molecular weight of PEG ranges between 350 and 3000 Da, and the carbon chain of the anchored lipid lies between 13 and 18 carbon. Multiple literature reports indicated that a higher molecular weight of PEG and a longer lipid chain increase nanoparticle circulation time and reduce immune cell uptake.

As the PEG-lipid dissociates from the LNP surface, it decreases the circulation time of the LNP. It provides more chances for delivering the mRNA cargo into target cells by an effect called “PEG-Dilemma.” In some instances, the molar % of the PEG-lipid is maintained at 1.5%. The in vivo transfection level was found to be independent of the carbon chain length of the lipid. An added advantage of PEG-lipids relies on their capability of conjugating a specific ligand to the LNP, thus aiding in targeted drug delivery.

### Helper Lipids

The primary function of helper lipids in the formulation of LNPs lies in supporting their stability during storage and in vivo circulation. Chemically, these are glycerolipids and non-cationic. Among the various helper lipids, sterols and phospholipids are the most widely used. Cholesterol is a natural component present in cell membranes. It is an exchangeable moiety that can be quickly accumulated in the LNP. Different studies have indicated that cholesterol might be present on the surface, within the lipid bilayer, or even conjugated with the ionized lipid within its core. It is usually incorporated in LNP formulation to maintain stability by filling gaps between lipids. Cholesterol is needed to regulate the density, uptake, and fluidity of the lipid bilayer matrix within the LNP.

Therefore, it controls the rigidity and integrity of the membrane, thereby preventing any leaks by the “condensing effect.” The hydrophobic tail, sterol ring flexibility, and polarity of hydroxy groups in cholesterol were reported to impact the efficacy of LNP delivery. Cholesterol also improves the circulation half-life of LNPs by reducing the surface-bound protein. Moreover, it helps by fusing with the endosomal membrane during the cellular uptake of LNPs. It plays a vital role in lowering the temperature needed for transitioning from the lamellar phase to the hexagonal phase; therefore, the mRNA cargo from the LNP will be delivered to the cytosol.

Including phospholipids in LNP formulation can help boost encapsulation (together with cholesterol) and increase cellular delivery. The number of phospholipids in the LNP is generally considerably reduced, increasing the cholesterol content for longer circulation times. Additionally, including phospholipids promotes the entrapment efficiency and transfection potency of the LNP. It has been reported that increasing the molar percentage of phospholipids contributes to expediting the efficacy of delivery by LNPs. These phospholipids in Zwitter ionic form have been reported to play a pivotal role in the assembly of the LNP through the stabilization of electrostatic interactions between the cationic lipid, mRNA cargo, and surrounding water molecules. However, the actual role of phospholipids in the delivery of mRNA via LNPs is still ambiguous.

### DETAILS OF THE INVENTION

Messenger RNA (mRNA) is crucial in translating genetic information from DNA into proteins. It includes several key components, each with a specific function. The 5′ Cap, a modified guanine nucleotide at the mRNA's 5′ end, ensures mRNA stability and aids in translation initiation, protecting the mRNA from degradation and assisting in ribosome recognition. Adjacent to the cap is the 5′ Untranslated Region (5′ UTR), a sequence not coding for protein but crucial in regulating translation efficiency and ribosome binding.

Advantageously, mRNA can be manufactured in a large-scale fashion and enables the production of a robust immune response based on mRNA encoding, for example, antigens that produce antibodies specific to proteins of the target infecting cell.

In various embodiments, the coding mRNA comprises, preferably in 5′- to 3′-direction, the following elements:
5′-cap structure selected from m7G(5′), m7G(5′)ppp(5′)(20MeA), or m7G(5′)ppp(5′)(2′OMeG);
The 5′-terminal start element is selected from AUG, GUG, and UUG.
3-UTR
An open reading frame to express RNA
poly(A) sequence comprising about 50 to about 250 adenosines.

The 5′ end of the mRNA contains a 7-methylguanosine (m7G) moiety, followed by a triphosphate moiety to the first nucleotide (m7GpppN). m7GpppN is called a 5′ cap, a protective structure that protects RNA from exonuclease cleavage, regulates pre-mRNA splicing, and initiates mRNA translation and nuclear export of the mRNA to the cytoplasm. The 5′ cap is also essential in recognizing non-self mRNA or exogenous mRNA from self mRNA or the endogenous mRNA by the innate immune system.

The mRNA can be modified to improve its efficacy and stability by introducing many post-transcriptional modifications. Some of these include 2′-O-methylation at position 2′ of the ribose ring at the first nucleotide (Cap 1, m7GpppNlm) and the second nucleotide e (Cap 2, m7GpppN1mN2m) as well. These modifications in the 5′ cap structure not only increase the translation efficiency of mRNA but also stop the activation of endosomal and cytosolic receptors, including RIG-I and MDA5, which act as defensive mechanisms against viral mRNA.

Hence, the 2′-O-methylation of the 5′ cap structure is a highly desirable property for increasing and enhancing the protein production from the mRNA after its transcription and blocking any undesirable immune responses from the host immune system to the antigenic IVT mRNA. This 5′ cap can be achieved by adding S-adenosyl methionine and the Cap 0 structure to the IVT mRNA reaction, which yields IVT mRNA with the Cap 1 structure and S-adenosyl-L-homocysteine. Cap 1 refers to m7GpppNm, where Nm represents any nucleotide with a 2′O methylation. This structure plays a crucial role in RNA stability and the initiation of protein synthesis. m7G represents a 7-methylguanosine residue. It's a modified guanine nucleotide with a methyl group attached to the nitrogen at the 7th position. This modification is crucial for RNA stability and efficient translation; ppp is a triphosphate bridge. It connects the 5′ end of the mRNA with the m7G cap. This linkage is unusual because it's a 5′-to-5′ triphosphate linkage, unlike the typical 5′-to-3′ phosphodiester bonds in RNA; Am signifies a 2′-O-methyladenosine residue. It's a modification where a methyl group is added to the 2′ hydroxyl group of the first nucleotide of the mRNA adjacent to the cap. This modification can enhance the stability of the mRNA and also plays a role in distinguishing self-RNA (e.g., from a cell's genes) from non-self-RNA (e.g., viruses or other pathogens) in the immune response.

The cap1 structure (m7GpppAm) is a common feature in eukaryotic mRNA and is essential for various aspects of RNA metabolism, including RNA stability, export from the nucleus, and translation initiation. It also helps recognize the mRNA by the ribosome and other components of the translation machinery. In the modified structure, there's an additional methyl group at the 3′ position of the m7G cap (m7G+m3′). This modification might further influence the interaction of the cap with cellular proteins and potentially affect mRNA stability and translation efficiency.

An example of the modified 5′-cap1 structure (m7G+m3′-5′-ppp-5′-Am) can be found in certain messenger RNAs (mRNAs) used in mRNA-based vaccines, such as those developed for COVID-19. In these vaccines, the mRNA carries the instructions to produce a specific viral protein (like the spike protein of the SARS-COV-2 virus) that triggers an immune response in the body. The modified cap structure plays a crucial role in these mRNA molecules.

The various types of CAPs that can be beneficial in the present invention include ARCA, Bridged Cap (BCAP), Cap0, Cap1, Cap2, Cap3, Cap4, CleanCap, Hypermodified Caps, Modified Cap1, Synthetic or Designer Caps, Tobacco Mosaic Virus (TMV) Cap, and Viral Caps.

In preferred embodiments, the cap1 structure of the coding mRNA of the invention is formed using co-transcriptional capping using tri-nucleotide cap analogs m7G(5′)ppp(5′)(2′OMeA)pG or m7G(5′)ppp(5′)(2′OMeG)pG. A preferred cap1 analog that may suitably be used in manufacturing the coding mRNA of the invention is m7G(5′)ppp(5′)(2′OMeA)pG.

The invention's open reading frame or coding sequence of mRNA can be prepared using any method known in the art, including chemical synthesis, such as, e.g. solid phase mRNA synthesis, and in vitro methods, such as mRNA in vitro transcription reactions.

In a preferred embodiment, the coding mRNA, preferably the mRNA, is obtained by mRNA in vitro transcription.

In embodiments, a typical selection of the components of an mRNA vaccine is given in Table 1.

TABLE 1
mRNA Vaccine design

| Element | Description | Position |
|---|---|---|
| Cap (2) | A modified 5′-cap1 structure | 1-2 |
| | (m7G + m3′-5′-ppp-5′-Am): GA | |
| 5′-UTR (52) | The 5′-untranslated region | 3-54 |
| | derived from human alpha-globin | |
| | RNA with an optimized Kozak | |
| | sequence. | |
| | GAAΨAAACΨAGΨAΨΨCΨΨCΨGGΨCCCCACAG | |
| | ACΨCAGAGAGAACCCGCCACC | |
| Signal | S glycoprotein signal peptide | 55-102 |
| peptide | (extended leader sequence) | |
| (48) | guides translocation of the | |
| | nascent polypeptide chain into | |
| | the endoplasmic reticulum: | |
| | AΨGΨΨCGΨGΨΨCCΨGGΨGCΨGCΨGCCΨCΨGG | |
| | ΨGΨCCAGCCAGΨGΨGΨG | |
| Coding region | Codon-optimized sequence: | +xxx |
| (stop codons | | |
| underlined) | | |
| (xxx) | | |
| 3′-UTR (268) | The 3′ untranslated region | +268 |
| | comprises two sequence elements | |
| | derived from the amino-terminal | |
| | enhancer of split (AES) mRNA | |
| | and the mitochondrial encoded | |
| | 12S ribosomal RNA to confer | |
| | RNA stability and high total | |
| | protein expression: | |
| | GCΨAGVΨGCCCCΨΨΨCCCGΨCCΨGGGΨACCCCGAGΨCΨ | |
| | CCCCCGACCΨCGGGΨCCCAGGΨAΨGCΨCCCACCΨCCAC | |
| | CΨGCCCCACΨCACCACCΨCΨGCΨAGΨΨCCAGACACCΨC | |
| | CCAAGCACGCAGCAAΨGCAGCΨCAAAACGCΨΨAGCCΨA | |
| | GCCACACCCCCACGGGAAACAGCAGΨGAΨΨAACCΨΨΨA | |
| | GCAAΨAAACGAAAGΨΨΨAACΨAAGCΨAΨACΨAACCCC | |
| | AGGGΨΨGGΨCAAΨΨΨCGΨGCCAGCCACACCCΨGGAGC | |
| | ΨAGC | |
| poly(A) (110) | A 110-nucleotide poly(A)-tail | +110 |
| | consisting of a stretch of 30 | |
| | adenosine residues, followed by | |
| | a 10-nucleotide linker sequence | |
| | and another 70 adenosine residues: | |
| | AAAAAAAAAAAAAAAAAAAAAAAAAAAAAAGCAΨAΨG | |
| | ACΨAAAAAAAAAAAAAAAAAAAAAAAAAAAAAAAAAA | |
| | AAAAAAAAAAAAAAAAAAAAAAAAAAAAAAAAAAAA | |

In embodiments, the nucleotide mixture used in mRNA in vitro transcription may additionally contain modified nucleotides as defined herein. Modifying codons, particularly in codon optimization, involves various strategies to enhance gene expression or protein synthesis in a target organism. Here's a list of common modifications applied to codons:

In codon optimization, structural changes to the RNA sequence are often made to enhance the stability and efficiency of mRNA translation. These changes are designed to avoid hindrances in the translation process and improve overall protein expression. Some common examples of such structural changes include:
Reducing Secondary Structure Formation: Altering sequences that form stable hairpin or stem-loop structures in the mRNA. For instance, a sequence like GGGGGG, which might form a strong secondary structure, could be altered to a less self-complementary sequence like GAGAGA without changing the amino acid sequence.
Adjusting GC Content: Modifying the GC content of the mRNA to optimize stability and efficiency. High GC content can lead to secondary solid structures, while low GC content might reduce mRNA stability. Adjustments are made to reach an optimal balance. For example, replacing AT-rich codons with GC-rich synonymous codons or vice versa.
Eliminating Motifs that Cause mRNA Degradation: Removing or altering sequences that are known to signal for rapid mRNA degradation. For example, specific sequences like AU-rich elements in eukaryotes might be modified to increase the half-life of the mRNA.
Avoiding Ribosomal Stalling Sequences: Changing sequences that can cause the ribosome to stall during translation. For instance, a stretch of rare codons or a sequence that forms a tight secondary structure might be modified to ensure smooth progression of the ribosome.
Removing Unintended Regulatory Elements: Altering sequences that could mimic regulatory elements like promoters, enhancers, or internal ribosome entry sites (IRES), which could interfere with proper transcription and translation.
Balancing tRNA Demand: Adjusting codon usage to match the tRNA pool of the host organism. Overusing a particular codon can deplete its corresponding tRNA, slowing translation. The sequence is modified to use more abundant tRNAs.
Minimizing Repeat Sequences: Reducing repetitive sequences that can lead to recombination events or genomic instability. This also helps in avoiding slippage during transcription or translation.

These structural changes are tailored to the specific requirements of the host organism and the protein being expressed. The goal is to create an mRNA sequence that is efficiently translated with minimal interruptions or instability, leading to higher protein yields.

In that context, preferred modified nucleotides comprise pseudouridine, N1-methylpseudouridine, 5-methylcytosine, and 5-methoxyuridine. Embodiments of uracil nucleotides in the nucleotide mixture are replaced (either wholly or partially) by pseudouridine and/or N1-methyl pseudouridine to obtain a modified coding mRNA.

In preferred embodiments, the nucleotide mixture (i.e., the fraction of each nucleotide in the mix) used for mRNA in vitro transcription reactions may be optimized for the given mRNA sequence.

In a further preferred embodiment, the coding mRNA, particularly the purified coding mRNA, is lyophilized. The mRNA of the invention, particularly the purified mRNA, may also be dried using spray-drying or spray-freeze drying.

In preferred embodiments, the coding mRNA of the invention is a purified mRNA, particularly purified mRNA that has a higher purity after specific purification steps (e.g., HPLC, TFF, Oligo d (T) purification, precipitation steps) than the starting material (e.g., in vitro transcribed mRNA). Typical impurities that are essentially not present in purified mRNA comprise peptides or proteins (e.g., enzymes derived from DNA-dependent mRNA in vitro transcription, e.g., mRNA polymerases, mRNAses, pyrophosphatase, restriction endonuclease, DNase), spermidine, BSA, abortive mRNA sequences, mRNA fragments (short double-stranded mRNA fragments, abortive sequences, etc.), free nucleotides (modified nucleotides, conventional NTPs, cap analog), template DNA fragments, buffer components (HEPES, TRIS, MgCI₂) etc. Other potential impurities derived from, e.g., fermentation procedures, comprise bacterial impurities (bioburden, bacterial DNA) or impurities derived from purification procedures (organic solvents, etc.).

### Pharmaceutical Vaccine

A second aspect relates to a vaccine comprising at least one coding mRNA of the first aspect.

Notably, embodiments relating to the vaccine of the second aspect may likewise be read on and be understood as suitable embodiments of the vaccine of the third aspect. Also, embodiments relating to the vaccine of the third aspect may likewise be read on and be understood as suitable embodiments of the vaccine of the second aspect (comprising the mRNA of the first aspect). In preferred embodiments of the second aspect, said vaccine comprises at least one mRNA encoding peptides or proteins according to the first aspect, or an immunogenic fragment or immunogenic variant thereof, wherein said vaccine is to be, preferably, administered intramuscularly or intradermal.

Preferably, intramuscular or intradermal administration of the said vaccine results in the expression of the encoded antigen in a subject. Preferably, the vaccine of the second aspect is suitable for an ideal vaccine.

The vaccine may comprise a safe and effective amount of the mRNA to result in the encoded antigenic protein's expression and activity. At the same time, a “safe and effective amount” is small enough to avoid serious side effects.

In the context of the invention, a “vaccine” refers to any type of vaccine in which the specified ingredients (e.g., mRNA encoding proteins or peptides, e.g., in association with a polymeric carrier or LNP), may be incorporated, optionally along with any further constituents, usually with at least one pharmaceutically acceptable carrier or excipient. The vaccine may be a dry vaccine, such as a powder or granules, or a solid unit, such as a lyophilized form. Alternatively, the vaccine may be liquid, and each constituent may be independently incorporated in dissolved or dispersed (e.g., suspended or emulsified) form.

In a preferred embodiment of the second aspect, the vaccine comprises mRNA coding at least one protein or peptide and, optionally, at least one pharmaceutically acceptable carrier or excipient.

In particularly preferred embodiments of the second aspect, the vaccine comprises at least one coding mRNA, wherein the coding mRNA includes or consists of an mRNA sequence that is identical or at least 70% to 99% to a nucleic acid sequence selected from the group consisting of epitopes chosen (Table 1), and, optionally, at least one pharmaceutically acceptable carrier or excipient.

The term “pharmaceutically acceptable carrier” or “pharmaceutically acceptable excipient” as used herein preferably includes the liquid or non-liquid basis of the vaccine for administration. If the vaccine is liquid, the carrier may be water, e.g., pyrogen-free water; isotonic saline or buffered (aqueous) solutions, e.g., phosphate, citrate, etc. buffered solutions. Water or preferably a buffer, more preferably an aqueous buffer, may be used, containing a sodium salt, preferably at least 50 mM of a sodium salt, a calcium salt, preferably at least 0.01 mM of a calcium salt, and optionally a potassium salt, preferably at least 3 mM of a potassium salt. According to preferred embodiments, the sodium, calcium, and, optionally, potassium salts may occur in the form of their halogenides, e.g., chlorides, iodides, or bromides, in the form of their hydroxides, carbonates, hydrogen carbonates, or sulfates, etc.

Furthermore, organic anions of the cations may be in the buffer. Accordingly, in embodiments, the mRNA vaccine of the invention may comprise pharmaceutically acceptable carriers or excipients using one or more pharmaceutically acceptable carriers or excipients to, e.g., increase stability, increase cell transfection, permit the sustained or delayed, increase the translation of encoded epitopes. In addition to traditional excipients such as all solvents, dispersion media, diluents, or other liquid vehicles, dispersion or suspension aids, surface active agents, isotonic agents, thickening or emulsifying agents, preservatives, excipients of the present invention can include, without limitation, lipidoids, liposomes, lipid nanoparticles, polymers, lipoplexes, core-shell nanoparticles, peptides, proteins, cells transfected with polynucleotides, hyaluronidase, nanoparticle mimics and combinations thereof. In embodiments, one or more compatible solid or liquid fillers, diluents, or encapsulating compounds, which are suitable for administration to a subject, may also be used.

The term “compatible,” as used herein, means that the constituents of the vaccine are capable of being mixed with mRNA and, optionally, a plurality of mRNAs of the vaccine in such a manner that no interaction occurs, which would substantially reduce the biological activity or the pharmaceutical effectiveness of the vaccine under typical use conditions (e.g., intramuscular, or intradermal administration).

Pharmaceutically acceptable carriers or excipients must have sufficiently high purity and sufficiently low toxicity to make them suitable for administration to a subject to be treated. Compounds that may be used as pharmaceutically acceptable carriers or excipients may be sugars, such as, for example, lactose, glucose, trehalose, mannose, and sucrose; starches, such as, for example, corn starch or potato starch; dextrose; cellulose and its derivatives, such for example, sodium carboxymethylcellulose, ethyl cellulose, cellulose acetate; powdered tragacanth; malt; gelatin; tallow; solid glidants, such as, for example, stearic acid, magnesium stearate; calcium sulfate; vegetable oils, such as, for instance, groundnut oil, cottonseed oil, sesame oil, olive oil, corn oil and oil from Theobroma; polyols, such as, for example, polypropylene glycol, glycerol, sorbitol, mannitol and polyethylene glycol; alginic acid.

At least one pharmaceutically acceptable carrier or excipient of the vaccine may preferably be selected to be suitable for intramuscular or intradermal delivery of the said vaccine. Accordingly, the vaccine is preferably a pharmaceutical vaccine suitable for intramuscular or intradermal administration.

The pharmaceutical vaccine is contemplated for use, but is not limited to, humans and other primates; mammals, including commercially relevant mammals such as cattle, pigs, horses, sheep, cats, dogs, mice, and rats; and birds, including commercially relevant birds such as poultry, chickens, ducks, geese, and turkeys.

Pharmaceutical vaccines of the present invention may suitably be sterile and pyrogen-free. Furthermore, one or more compatible solid or liquid filler diluents or encapsulating compounds, which are suitable for administration to a person, may also be used. The term “compatible,” as used herein, means that the constituents of the vaccine are capable of being mixed with mRNA and, optionally, the further coding mRNA of the vaccine in such a manner that no interaction occurs, which would substantially reduce the biological activity or the pharmaceutical effectiveness of the vaccine under typical use conditions.

In embodiments, the vaccine as defined herein may comprise a plurality or at least more than one of the coding mRNA species as defined in the context of the first aspect of the invention.

In a preferred embodiment of the second aspect, the mRNA is complexed or associated with to obtain a formulated vaccine. A formulation in that context may have the function of adjuvant. A formulation in that context may also have the function of protecting the coding mRNA from degradation.

In a preferred embodiment of the second aspect, the coding mRNA is complexed or associated with or at least partially complexed or partially associated with one or more cationic or polycationic compounds, preferably cationic or polycationic polymer, cationic or polycationic polysaccharide, cationic or polycationic lipid, cationic or polycationic protein, cationic or polycationic peptide, or any combinations thereof.

The term “cationic or polycationic compound” as used herein will be recognized and understood by the person of ordinary skill in the art and is, for example, intended to refer to a charged molecule, which is positively charged at a pH value ranging from about 1 to 9. Accordingly, a cationic component, e.g., a cationic peptide, cationic protein, cationic polymer, cationic polysaccharide, or cationic lipid, may be any positively charged compound or polymer that is positively charged under physiological conditions. A “cationic or polycationic peptide or protein” may contain at least one positively charged amino acid or more than one positively charged amino acid, e.g., selected from Arg, His, Lys, Glu, Asp, or Orn. Accordingly, “polycationic” components are also within the scope of exhibiting more than one positive charge under the given conditions.

Cationic or polycationic compounds, being particularly preferred in this context, may be selected from the following list of cationic or polycationic peptides or proteins of fragments thereof: protamine, nucleoline, spermine or spermidine, or other cationic peptides or proteins, such as poly-L-lysine (PLL), poly-arginine, essential polypeptides, cell-penetrating peptides (CPPs), including HIV-binding peptides, HIV-1 Tat (HIV), Tat-derived peptides, Penetratin, VP22 derived or analog peptides, HSV VP22 (Herpes simplex), MAP, KALA or protein transduction domains (PTDs), PpT620, proline-rich peptides, arginine-rich peptides, lysine-rich peptides, MPG-peptide(s), Pep-1, L-oligomers, Calcitonin peptide(s), Antennapedia-derived peptides, pAntp, plsl, FGF, Lactoferrin, Transportan, Buforin-2, Bac715-24, SynB, SynB(1), pVEC, hCT-derived peptides, SAP, or histones. The nucleic acid, as defined herein, preferably the mRNA as defined herein, is more complex with one or more polycations, preferably with protamine or oligofectamine, most preferably with protamine.

Coding mRNA is complexed with protamine in a preferred embodiment of the second aspect.

In this context, it is particularly preferred that coding mRNA is complexed or at least partially complexed with a cationic or polycationic compound and a polymeric carrier, preferably cationic proteins or peptides.

In a preferred embodiment of the second aspect, the vaccine comprises at least one coding mRNA complexed with one or more cationic or polycationic compounds, preferably protamine, and at least one free coding mRNA. In this context, at least one coding mRNA is particularly preferred to be complexed or at least partially complexed with protamine. Preferably, the molar ratio of the nucleic acid, particularly the mRNA of the protamine-complexed mRNA to the free mRNA, may be selected from a molar ratio of about 0.001:1 to about 1:0.001, including a ratio of about 1:1. Suitably, the complexed mRNA is complexed with protamine by addition of protamine-trehalose solution to the mRNA sample at a mRNA: protamine weight to weight ratio (w/w) of 2:1.

In a preferred embodiment of the second aspect, coding mRNA is complexed or partially complexed, with at least one cationic or polycationic protein, peptide, or a combination thereof.

According to embodiments, the vaccine of the present invention comprises the coding mRNA as defined in the context of the first aspect and a polymeric carrier.

The term “polymeric carrier,” as used herein, will be recognized, and understood by the person of ordinary skill in the art and is, e.g., intended to refer to a compound that facilitates transport and complexation of another compound (e.g., cargo mRNA). A polymeric carrier is typically a carrier that is formed of a polymer. A polymeric carrier may be associated with its cargo (e.g., coding mRNA) by covalent or non-covalent interaction. A polymer may be based on different subunits, such as a copolymer.

Suitable polymeric carriers in that context may include, for example, polyacrylates, polyalkycyanoacrylates, polylactide, polylactide-polyglycolide copolymers, polycaprolactones, dextran, albumin, gelatin, alginate, collagen, chitosan, cyclodextrins, protamine, PEGylated protamine, PEGylated PLL and polyethylenimine (PEI), dithiobis(succinimidylpropionate) (DSP), Dimethyl-3,3′-dithiobispropionimidate (DTBP), polyethylene imine biscarbamate (PEIC), poly(L-lysine) (PLL), histidine modified PLL, poly(N-vinylpyrrolidone) (PVP), poly(propylenimine (PPI), poly(amidoamine) (PAMAM), poly(amido ethylenimine) (SS-PAEI), triehtylenetetramine (TETA), poly-aminoester, poly(4-hydroxy-L-proine ester) (PHP), poly(allylamine), poly(a-[4-aminobutyl]-L-glycolic acid (PAGA), Poly(D,L-lactic-co-glycolid acid (PLGA), Poly(N-ethyl-4-vinylpyridinium bromide), poly(phosphazene)s (PPZ), poly(phosphoester)s (PPE), poly(phosphoramidate)s (PPA), poly(N-2-hydroxypropylmethacrylamide) (pHPMA), poly(2-(dimethylamino)ethyl methacrylate) (pDMAEMA), poly(2-aminoethyl propylene phosphate) PPE_EA), galactosylated chitosan, N-dodecylated chitosan, histone, collagen and dextran-spermine. In one embodiment, the polymer may be an inert polymer such as, but not limited to, PEG. In one embodiment, the polymer may be cationic, such as, but not limited to, PEI, PLL, TETA, poly(allylamine), Poly(N-ethyl-4-vinylpyridinium bromide), pHPMA, and pDMAEMA. In one embodiment, the polymer may be a biodegradable PEI such as, but not limited to, DSP, DTBP, and PEIC. In one embodiment, the polymer may be biodegradable, such as, but not limited to, histidine-modified PLL, SS-PAEI, poly\hat{ }-aminoester), PHP, PAGA, PLGA, PPZ, PPE, PPA and PPE-EA.

When PEI is present, it may be branched PEI of a molecular weight ranging from 10 to 40 kDa, e.g., 25 kDa branched PEI (Sigma #408727). In some embodiments, the polymer-based nanoparticle comprises PEI. In some embodiments, the PEI is branched PEI. PEI may be a branched PEI of a molecular weight ranging from 10 to 40 kDa, e.g., 25 kDa. In some embodiments, the PEI is linear. In some embodiments, the nanoparticle has a size of or less than about 60 nm (e.g., of or less than about 55 nm, of or less than about 50 nm, of or less than about 45 nm, of or less than about 40 nm, of or less than about 35 nm, of or less than about 30 nm, or of or less than about 25 nm). Suitable nanoparticles may range from 25 to 60 nm, e.g., 30 to 50 nm.

A suitable polymeric carrier may be a polymeric carrier formed by disulfide-crosslinked cationic compounds. The disulfide-crosslinked cationic compounds may be the same or different from each other. The polymeric carrier can also contain further components. The polymeric carrier used in the present invention may comprise mixtures of cationic peptides, proteins, polymers, and optionally further components as defined herein, which are crosslinked by disulfide bonds (via-SH groups).

In a particularly preferred embodiment, the polymeric carrier is a peptide polymer, preferably a polyethylene glycol/peptide polymer as defined above, and a lipid component, preferably a lipidoid component. In a preferred embodiment of the second aspect, coding mRNA of the first aspect is complexed or associated with a polymeric carrier, preferably with a polyethylene glycol/peptide polymer as defined above, and a lipidoid component, wherein the lipidoid component is a compound.

According to preferred embodiments, the peptide polymer comprises preferably lipidoid 3-C12-OH, which is used to complex coding mRNA of the first aspect to form complexes having an N/P ratio from about 0.1 to about 20, or from about 0.2 to about 15, or from about 2 to about 15, or from about 2 to about 12, wherein the N/P ratio is defined as the mole ratio of the nitrogen atoms of the basic groups of the cationic peptide or polymer to the phosphate groups of the nucleic acid.

In preferred embodiments of the second aspect, coding mRNA is complexed, encapsulated, partially encapsulated, or associated with one or more lipids (e.g., cationic lipids and/or neutral lipids), thereby forming liposomes, lipid nanoparticles (LNPs), lipoplexes, and nanoliposomes.

The liposomes, lipid nanoparticles (LNPs), lipoplexes, and nanoliposomes—incorporated mRNA may be entirely or partially located in the interior space of the liposomes, lipid nanoparticles (LNPs), lipoplexes, and nanoliposomes, within the membrane, or associated with the exterior surface of the membrane. Incorporating nucleic acid into liposomes is also referred to herein as “encapsulation,” wherein the mRNA is entirely contained within the interior space of the liposomes, lipid nanoparticles (LNPs), lipoplexes, and nanoliposomes. The purpose of incorporating an mRNA into liposomes, lipid nanoparticles (LNPs), lipoplexes, and nanoliposomes is to protect the mRNA from an environment that may contain enzymes or chemicals that degrade mRNA and systems or receptors that cause the rapid excretion of the mRNA. Moreover, incorporating an mRNA into liposomes, lipid nanoparticles (LNPs), lipoplexes, and nanoliposomes may promote the uptake of the mRNA and, hence, enhance the therapeutic effect of the mRNA-encoding antigenic peptides. Accordingly, incorporating an mRNA into liposomes, lipid nanoparticles (LNPs), lipoplexes, and nanoliposomes may be particularly suitable for a vaccine, e.g., intramuscular or intradermal administration.

In this context, “complexed” or “associated” refers to the essentially stable combination of coding mRNA of the first aspect with one or more lipids into larger complexes or assemblies without covalent binding.

The term “lipid nanoparticle,” also referred to as “LNP,” is not restricted to any morphology and includes any morphology generated when a cationic lipid and optionally one or more further lipids are combined, e.g., in an aqueous environment and the presence of mRNA. For example, a liposome, a lipid complex, a lipoplex, and the like are within the scope of a lipid nanoparticle (LNP).

Liposomes, lipid nanoparticles (LNPs), lipoplexes, and nanoliposomes can be of different sizes, such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter.

LNPs of the invention are suitably characterized as microscopic vesicles having an interior aqua space sequestered from an outer medium by a membrane of one or more bilayers. Bilayer membranes of LNPs are typically formed by amphiphilic molecules, such as lipids of synthetic or natural origin, that comprise spatially separated hydrophilic and hydrophobic domains. Bilayer membranes of the liposomes can also be formed by amphophilic polymers and surfactants (e.g., polymerosomes, niosomes, etc.). In the context of the present invention, an LNP typically serves to transport the mRNA of the first aspect to a target tissue.

Accordingly, in preferred embodiments of the second aspect, mRNA is complexed with one or more lipids, forming lipid nanoparticles (LNP), wherein said LNP is particularly suitable for intramuscular and intradermal administration.

LNPs typically comprise a cationic lipid and one or more excipients selected from neutral lipids, charged lipids, steroids, and polymer-conjugated lipids (e.g., PEGylated lipids). The coding mRNA may be encapsulated in the lipid portion of the LNP or an aqueous space enveloped by some or the entire lipid portion of the LNP. The coding mRNA or a portion thereof may also be associated and complexed with the LNP. An LNP may comprise any lipid capable of forming a particle to which the nucleic acids are attached or in which one or more nucleic acids are encapsulated. Preferably, the LNP comprising nucleic acids comprises one or more cationic lipids and stabilizing lipids. Stabilizing lipids include neutral lipids and PEGylated lipids.

The cationic lipid of an LNP may be cationisable, i.e., it becomes protonated as the pH is lowered below the pKa of the ionizable group of the lipid but is progressively more neutral at higher pH values. At pH values below the pKa, the lipid can be associated with negatively charged nucleic acids. In certain embodiments, the cationic lipid comprises a zwitterionic lipid that assumes a positive charge on pH decrease.

The LNP may comprise any further cationic or cationisable lipid, i.e., any of several lipid species that carry a net positive charge at a selective pH, such as physiological pH.

Such lipids include, but are not limited to, DSDMA, N, N-dioleyl-N,N-dimethylammonium chloride (DODAC), N,N-distearyl-N,N-dimethylammonium bromide (DDAB), 1,2-dioleoyltrimethyl ammonium propane chloride (DOTAP) (also known as N-(2,3-dioleoyloxy)propyl)-N,N,N-trimethylammonium chloride and 1,2-Dioleyloxy-3-trimethylaminopropane chloride salt, N-(2,3-dioleyloxy)propyl)-N,N,N-trimethylammonium chloride (DOTMA), N,N-dimethyl-2,3-dioleyloxy)propylamine (DODMA), ckk-E12, ckk, 1,2-Dilinoleyloxy-N,N-dimethylaminopropane (DLinDMA), 1,2-Dilinolenyloxy-N,N-dimethylaminopropane (DLenDMA), 1,2-di-y-linolenyloxy-N,N-dimethylaminopropane (g-DLenDMA), 98N12-5,1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA CI), ICE (Imidazol-based), HGT5000, HGT5001, DMDMA, CLinDMA, CpLinDMA, DMOBA, DOcarbDAP, DLincarbDAP, DLinCDAP, KLin-K-DMA, HGT4003, 1,2-Dilinoleoyl-3-trimethylaminopropane chloride salt (DLin-TAP CI), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DM A), 2,2-Dilinoleyl-4-dimethylaminomethyl-[1,3]-DLin-K-DMA) or analogs thereof, (3aR,5s,6aS)-N,N-dimethyl-2,2-di((9Z, 12Z)-octadeca-9,12-dienyl)tetrahydro-3aH-cyclopenta[d][1,3]dioxol-5-amine, (6Z,9Z,28Z,31 Z)-heptatriaconta-6,9,28,31-tetraen-19-yl-4-(dimethylamino)butanoate (MC3), ALNY-100 ((3aR,5s,6aS)-N,N-dimethyl-2,2-di((9Z, 12Z)-octadeca-9,12-dienyl)tetrahydro-3aH-cyclopenta[d] [1,3]dioxol-5-amine)), 1,1-(2-(4-(2-((2-(bis(2-hydroxydodecyl)amino)ethyl)(2-hydroxydodecyl)amino)ethyl)piperazin-1-yl)ethylazanediyl)didodecan-2-ol (C12-200), 2,2-dilinoleyl-4-(2-dimethylaminoethyl)-[1,3]-dioxolane (DLin-K-C2-DMA), 2,2-dilinoleyl-4-dimethylaminomethyl-[1,3]-dioxolane (DL in-K-DMA;XTC), NC98-5 (4,7,13-tris(3-oxo-3-(undecylamino)propyl)-NI,N 16-diundecyl-4,7,10,13-tetraazahexadecane-1,16-diamide), (6Z,9Z,28Z,31 Z)-heptatriaconta-6,9,28,31-tetraen-19-yl 4-(dimethylamino) butanoate (DLin-M-C3-DMA), 3-((6Z,9Z,28Z,31 Z)-heptatriaconta-6,9,28,31-tetraen-19-yloxy)-N,N-dimethylpropan-1-amine (MC3 Ether), 4-((6Z,9Z,28Z,31 Z)-heptatriaconta-6,9,28,31-tetraen-19-yloxy)-N,N-dimethylbutan-1-amine (MC4 Ether), LIPOFECTIN® (commercially available cationic liposomes comprising DOTMA and 1,2-dioleoyl-sn-3phosphoethanolamine (DOPE), from GIBCO/BRL, Grand Island, N Y.); LIPOFECTAMINE® (commercially available cationic liposomes comprising N-(1-(2,3dioleyloxy)propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.) or any combination of any of the foregoing.

In some embodiments, the lipid is selected from the 98N12-5, C12-200, and ckk-E12 groups.

In one embodiment, the further cationic lipid is an amino lipid including but not limited to 1,2-dilinoleyoxy-3-(dimethylamino)acetoxypropane (DLin-DAC), 1,2-dilinoleyoxy-3morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3dimethylaminopropane (DLin-2-DMAP), 1,2-dilinoleyloxy-3-trimethylaminopropane chloride salt (DLin-TMA.CI), 1,2-dilinoleoyl-3-trimethylaminopropane chloride salt (DLin-TAP.CI), 1,2-dilinoleyloxy-3-(N-methylpiperazino)propane (DLin-MPZ), 3-(N,Ndilinoleylamino)-1,2-propanediol (DLinAP), 3-(N, N-dioleylamino)-1,2-propanediol (DOAP), 1,2-dilinoleyloxo-3-(2-N, N-dimethylamino)ethoxypropane (DLin-EG-DMA), and 2,2-dilinoleyl-4-dimethylaminomethyl-[1,3]-dioxolane (DLin-K-DMA), 2,2-dilinoleyl-4-(2-dimethylaminoethyl)-[1,3]-dioxolane (DLin-KC2-DMA); dilinoleyl-methyl-4-dimethylaminobutyrate (DLin-MC3-DMA); MC3 (US20,100,324120).

According to further embodiments, the vaccine of the second aspect may comprise at least one adjuvant. Suitably, the adjuvant is preferably added to enhance the immunostimulatory properties of the vaccine.

The term “adjuvant” as used herein will be recognized and understood by the person of ordinary skill in the art and is, for example, intended to refer to a pharmacological and immunological agent that may modify, e.g., enhance, the effect of other agents (herein: the impact of the coding mRNA), or that may be suitable to support administration and delivery of the vaccine. The term “adjuvant” refers to a broad spectrum of substances. Typically, these substances can increase the immunogenicity of antigens. For example, adjuvants may be recognized by the innate immune systems and, e.g., may elicit an innate immune response (a nonspecific immune response).“Adjuvants” typically do not elicit an adaptive immune response. In the context of the invention, adjuvants may enhance the effect of the antigenic peptide or protein provided by the coding RNA. In that context, at least one adjuvant may be selected from any adjuvant known to a skilled person and suitable for the present case, i.e., supporting the induction of an immune response in a subject, e.g., a human subject.

The vaccine of the second aspect may comprise, besides the components specified herein, at least one further component which may be selected from the group consisting of further antigens (e.g., in the form of a peptide or protein) or further antigen-encoding nucleic acids, a further immunotherapeutic agent; one or more auxiliary substances (cytokines, such as monokines, lymphokines, interleukins or chemokines); or any further compound, which is known to be immune stimulating due to its binding affinity (as ligands) to human Toll-like receptors; and an adjuvant nucleic acid, preferably an immunostimulatory RNA (isRNA), e.g., CpG-RNA etc.

The LNP formulation is also an adjuvant.

### Vaccine

In a third aspect, the present invention provides a PD and related diseases vaccine wherein the vaccine comprises the coding RNA of the first aspect and, optionally, the vaccine of the second aspect.

Notably, embodiments relating to the vaccine of the second aspect may likewise be read on and be understood as suitable embodiments of the vaccine of the third aspect. Also, embodiments relating to the vaccine of the third aspect may likewise be read on and be understood as suitable embodiments of the vaccine of the second aspect (comprising the RNA of the first aspect).

The term “vaccine” will be recognized and understood by the person of ordinary skill in the art and is, for example, intended to be a prophylactic or therapeutic material providing at least one epitope or antigen, preferably an immunogen. In the context of the invention, the antigen or antigenic function is provided by the inventive coding RNA of the first aspect (said RNA comprising a coding sequence encoding an antigenic peptide or protein derived from proteins or peptides) or the vaccine of the second aspect (consisting of the RNA of the first aspect).

In preferred embodiments of the third aspect, the vaccine comprising the first aspect's mRNA, or the second aspect's vaccine elicits an adaptive immune response, preferably an adaptive immune response against PDs proteins known to cause the disease.

In preferred embodiments of the third aspect, the vaccine comprising the first aspect's RNA or the second aspect's vaccine induces strong humoral and cellular immune responses, both B-cell and preferably strong CD4+ and CD8+ T-cell responses.

According to a preferred embodiment of the third aspect, the vaccine, as defined herein, may further comprise a pharmaceutically acceptable carrier and optionally at least one adjuvant as specified in the context of the second aspect.

The vaccine can be used according to the invention for human medical and veterinary medical purposes (mammals, vertebrates, avian species), as a pharmaceutical vaccine, or as a vaccine.

Further, the present invention relates to the first medical use of the coding RNA of the first aspect, the vaccine of the second aspect, and the vaccine of the third aspect.

Accordingly, the RNA of the first aspect, the vaccine of the second aspect, and the vaccine of the third aspect are used as a medicament.

The present invention provides several applications and uses of the coding RNA of the first aspect, the vaccine of the second aspect, or the vaccine of the third aspect.

RNA vaccine, as vaccine may be used for human medical and veterinary medical purposes, preferably for human medical purposes.

Said RNA vaccine is used as a medicament for human medical purposes. The RNA, vaccine, and vaccine may suit any immunocompromised recipients,

In yet another aspect, the present invention relates to the second medical use of the coding RNA of the first aspect, the vaccine of the second aspect, and the vaccine of the third aspect.

In embodiments, the RNA of the first aspect, the vaccine of the second aspect, and the vaccine of the third aspect are for use in the treatment or prophylaxis of the accumulation of AB or tau protein or a disorder related to such an infection.

The vaccine or the vaccine as defined herein may preferably be administered locally. An intradermal, subcutaneous, intranasal, or intramuscular route may administer vaccine or vaccines. Inventive vaccines or vaccines of the invention are, therefore, preferably formulated in liquid (or sometimes in solid) form. In embodiments, conventional needle or needle-free jet injection may administer the inventive vaccine. Preferred in that context is the RNA, the vaccine, and the vaccine administered by intramuscular needle injection.

An mRNA sequence for an antibody against the α-Synuclein protein and its isomers and tau protein, as designed in this invention, is shown in Table 2, wherein the epitopes are optionally linked as a single chain.

TABLE 2
Residue Beta-Amyloid Fibril, Tau protein, α-Synuclein
protein, TDP-43 protein, and FUS protein sequence and
selected epitopes.
2MXU (PDB: 42-Residue Beta-Amyloid Fibril) (Aβ42)
Amino Acid Sequence (42)
DAEFRHDSGYEVHHQKLVFFAEDVGSNKGAIIGLMVGGVVIA (42 amino acids)
Epitopes: EFRHDSGYEVHH, EDVGSNKG
P10636 TAU_HUMAN (Tau Protein)
AA Sequence (758):
MAEPRQEFEVMEDHAGTYGLGDRKDQGGYTMHQDQEGDTDAGLKESPLQTPTEDGSE
EPGSETSDAKSTPTAEDVTAPLVDEGAPGKQAAAQPHTEIPEGTTAEEAGIGDTPSLEDE
AAGHVTQEPESGKVVQEGFLREPGPPGLSHQLMSGMPGAPLLPEGPREATRQPSGTGPE
DTEGGRHAPELLKHQLLGDLHQEGPPLKGAGGKERPGSKEEVDEDRDVDESSPQDSPPS
KASPAQDGRPPQTAAREATSIPGFPAEGAIPLPVDFLSKVSTEIPASEPDGPSVGRAKGQD
APLEFTFHVEITPNVQKEQAHSEEHLGRAAFPGAPGEGPEARGPSLGEDTKEADLPEPSE
KQPAAAPRGKPVSRVPQLKARMVSKSKDGTGSDDKKAKTSTRSSAKTLKNRPCLSPKH
PTPGSSDPLIQPSSPAVCPEPPSSPKYVSSVTSRTGSSGAKEMKLKGADGKTKIATPRGAA
PPGQKGQANATRIPAKTPPAPKTPPSSGEPPKSGDRSGYSSPGSPGTPGSRSRTPSLPTPPT
REPKKVAVVRTPPKSPSSAKSRLQTAPVPMPDLKNVKSKIGSTENLKHQPGGGKVQIIN
KKLDLSNVQSKCGSKDNIKHVPGGGSVQIVYKPVDLSKVTSKCGSLGNIHHKPGGGQV
EVKSEKLDFKDRVQSKIGSLDNITHVPGGGNKKIETHKLTFRENAKAKTDHGAEIVYKS
PVVSGDTSPRHLSNVSSTGSIDMVDSPQLATLADEVSASLAKQGL
Epitopes:
MAEPRQEFE, NAKAKTDHG, LREPGPPGLS, THVPGGGNKKI, HHKPGGGQVEVK,
TPNVQKEQAHSEEHL, SKIGSTENLKHQPGGGKV, SKCGSKDNIKHVPGGGSV,
TEIPASEPDGPSVGRAKGQDAP
P37840·SYUA_HUMAN (α-Synuclein)
Amino Acid Sequence (140):
MDVFMKGLSKAKEGVVAAAEKTKQGVAEAAGKTKEGVLYVGSKTKEGVVHGVATV
AEKTKEQVTNVGGAVVTGVTAVAQKTVEGAGSIAAATGFVKKDQLGKNEEGAPQEGI
LEDMPVDPDNEAYEMPSEEGYQDYEPEA
Epitopes:
KGLSK, EKTKQGVAEAAGKTKEGV, VGSKTKEGVV,
AEKTKEQVTNVGGAVVTG, TAVAQKTVE, FVKKDQLG
Q13148.TADBP_HUMAN (TDP-43)
Amino Acid Sequence (414)
MSEYIRVTEDENDEPIEIPSEDDGTVLLSTVTAQFPGACGLRYRNPVSQCMRGVRLVEGI
LHAPDAGWGNLVYVVNYPKDNKRKMDETDASSAVKVKRAVQKTSDLIVLGLPWKTT
EQDLKEYFSTFGEVLMVQVKKDLKTGHSKGFGFVRFTEYETQVKVMSQRHMIDGRWC
DCKLPNSKQSQDEPLRSRKVFVGRCTEDMTEDELREFFSQYGDVMDVFIPKPFRAFAFV
TFADDQIAQSLCGEDLIIKGISVHISNAEPKHNSNRQLERSGREGGNPGGFGNQGGFGNS
RGGGAGLGNNQGSNMGGGMNFGAFSINPAMMAAAQAALQSSWGMMGMLASQQNQ
SGPSGNNQNQGNMQREPNQAFGSGNNSYSGSNSGAAIGWGSASNAGSGSGFNGGFGSS
MDSKSSGWGM
Epitopes
RVTEDENDEPIEIPSEDDGTV, RYRNPVSQCMRGV, HAPDAGWGN, TTEQDLKEYFS,
ETQVKVMSQRHMIDG, PNSKQSQDEPLRS, DMTEDELREFFS, GEDLIIKG,
AEPKHNSNRQLERSGRFGGNPGGF, GFNGGFGSSMDSKSSG
P35637.FUS_HUMAN
Amino Acids (526)
MASNDYTQQATQSYGAYPTQPGQGYSQQSSQPYGQQSYSGYSQSTDTSGYGQSSYSSY
GQSQNTGYGTQSTPQGYGSTGGYGSSQSSQSSYGQQSSYPGYGQQPAPSSTSGSYGSSS
QSSSYGQPQSGSYSQQPSYGGQQQSYGQQQSYNPPQGYGQQNQYNSSSGGGGGGGGG
GNYGQDQSSMSSGGGSGGGYGNQDQSGGGGSGGYGQQDRGGRGRGGSGGGGGGGG
GGYNRSSGGYEPRGRGGGRGGRGGMGGSDRGGFNKFGGPRDQGSRHDSEQDNSDNN
TIFVQGLGENVTIESVADYFKQIGIIKTNKKTGQPMINLYTDRETGKLKGEATVSFDDPPS
AKAAIDWFDGKEFSGNPIKVSFATRRADFNRGGGNGRGGRGRGGPMGRGGYGGGGSG
GGGRGGFPSGGGGGGGQQRAGDWKCPNPTCENMNFSWRNECNQCKAPKPDGPGGGP
GGSHMGGNYGDDRRGGRGGYDRGGYRGRGGDRGGFRGGRGGGDRGGFGPGKMDSR
GEHRQDRRERPY
Epitopes:
LGENVTIESVADYFKQI, IIKTNKKTGQ, RETGKLK, FDGKEFSG

Table 3 lists the Open Reading Frames for the listed NDs.

TABLE 3
Open Reading Frames for mRNA Vaccines

| Disease | Aggregating | mRNA Open Reading Frame |
|---|---|---|
| Alzheimer′s | Aβ42, Tau | GAAUUUCGCCAUGAUAGCGGCUAUGAAGUGCAUCAU |
| Disease (AD) | | GGCAGCGGCAGCGGCAGCGGCAGCGAAGAUGUGGGC |
| GSGSGSGS | | AGCAACAAAGGCGGCAGCGGCAGCGGCAGCGGCAGC |
| | | AUGGCGGAACCGCGCCAGGAAUUUGAAGGCAGCGGC |
| | | AGCGGCAGCGGCAGCAACGCGAAAGCGAAAACCGAU |
| | | CAUGGCGGCAGCGGCAGCGGCAGCGGCAGCCUGCGC |
| | | GAACCGGGCCCGCCGGGCCUGAGCGGCAGCGGCAGC |
| | | GGCAGCGGCAGCACCCAUGUGCCGGGCGGCGGCAAC |
| | | AAAAAAAUUGGCAGCGGCAGCGGCAGCGGCAGCCAU |
| | | CAUAAACCGGGCGGCGGCCAGGUGGAAGUGAAAGGC |
| | | AGCGGCAGCGGCAGCGGCAGCACCCCGAACGUGCAG |
| | | AAAGAACAGGCGCAUAGCGAAGAACAUCUGGGCAGC |
| | | GGCAGCGGCAGCGGCAGCAGCAAAAUUGGCAGCACC |
| | | GAAAACCUGAAACAUCAGCCGGGCGGCGGCAAAGUG |
| | | GGCAGCGGCAGCGGCAGCGGCAGCAGCAAAUGCGGC |
| | | AGCAAAGAUAACAUUAAACAUGUGCCGGGCGGCGGC |
| | | AGCGUGGGCAGCGGCAGCGGCAGCGGCAGCACCGAA |
| | | AUUCCGGCGAGCGAACCGGAUGGCCCGAGCGUGGGC |
| | | CGCGCGAAAGGCCAGGAUGCGCCG |
| Amyotrophic | TDP-43, | CUGGGCGAAAACGUGACCAUUGAAAGCGUGGCGGAU |
| Lateral | FUS | UAUUUUAAACAGAUUGGCAGCGGCAGCGGCAGCGGC |
| Sclerosis | | AGCAUUAUUAAAACCAACAAAAAAACCGGCCAGGGC |
| (ALS) | | AGCGGCAGCGGCAGCGGCAGCCGCGAAACCGGCAAA |
| | | CUGAAAGGCAGCGGCAGCGGCAGCGGCAGCUUUGAU |
| | | GGCAAAGAAUUUAGCGGCGGCAGCGGCAGCGGCAGC |
| | | GGCAGCCGCGUGACCGAAGAUGAAAACGAUGAACCG |
| | | AUUGAAAUUCCGAGCGAAGAUGAUGGCACCGUGGGC |
| | | AGCGGCAGCGGCAGCGGCAGCCGCUAUCGCAACCCG |
| | | GUGAGCCAGUGCAUGCGCGGCGUGGGCAGCGGCAGC |
| | | GGCAGCGGCAGCCAUGCGCCGGAUGCGGGCUGGGGC |
| | | AACGGCAGCGGCAGCGGCAGCGGCAGCACCACCGAA |
| | | CAGGAUCUGAAAGAAUAUUUUAGCGGCAGCGGCAGC |
| | | GGCAGCGGCAGCGAAACCCAGGUGAAAGUGAUGAGC |
| | | CAGCGCCAUAUGAUUGAUGGCGGCAGCGGCAGCGGC |
| | | AGCGGCAGCCCGAACAGCAAACAGAGCCAGGAUGAA |
| | | CCGCUGCGCAGCGGCAGCGGCAGCGGCAGCGGCAGC |
| | | GAUAUGACCGAAGAUGAACUGCGCGAAUUUUUUAGC |
| | | GGCAGCGGCAGCGGCAGCGGCAGCGGCGAAGAUCUG |
| | | AUUAUUAAAGGCGGCAGCGGCAGCGGCAGCGGCAGC |
| | | GCGGAACCGAAACAUAACAGCAACCGCCAGCUGGAA |
| | | CGCAGCGGCCGCUUUGGCGGCAACCCGGGCGGCUUU |
| | | GGCAGCGGCAGCGGCAGCGGCAGCGGCUUUAACGGC |
| | | GGCUUUGGCAGCAGCAUGGAUAGCAAAAGCAGCGGC |
| Corticobasal | Tau | AUGGCGGAACCGCGCCAGGAAUUUGAAGGCAGCGGC |
| Degeneration | | AGCGGCAGCGGCAGCAACGCGAAAGCGAAAACCGAU |
| (CBD) | | CAUGGCGGCAGCGGCAGCGGCAGCGGCAGCCUGCGC |
| | | GAACCGGGCCCGCCGGGCCUGAGCGGCAGCGGCAGC |
| | | GGCAGCGGCAGCACCCAUGUGCCGGGCGGCGGCAAC |
| | | AAAAAAAUUGGCAGCGGCAGCGGCAGCGGCAGCCAU |
| | | CAUAAACCGGGCGGCGGCCAGGUGGAAGUGAAAGGC |
| | | AGCGGCAGCGGCAGCGGCAGCACCCCGAACGUGCAG |
| | | AAAGAACAGGCGCAUAGCGAAGAACAUCUGGGCAGC |
| | | GGCAGCGGCAGCGGCAGCAGCAAAAUUGGCAGCACC |
| | | GAAAACCUGAAACAUCAGCCGGGCGGCGGCAAAGUG |
| | | GGCAGCGGCAGCGGCAGCGGCAGCAGCAAAUGCGGC |
| | | AGCAAAGAUAACAUUAAACAUGUGCCGGGCGGCGGC |
| | | AGCGUGGGCAGCGGCAGCGGCAGCGGCAGCACCGAA |
| | | AUUCCGGCGAGCGAACCGGAUGGCCCGAGCGUGGGC |
| | | CGCGCGAAAGGCCAGGAUGCGCCG |
| Frontotemporal | Tau, TDP- | AUGGCGGAACCGCGCCAGGAAUUUGAAGGCAGCGGC |
| Dementia | 43, FUS | AGCGGCAGCGGCAGCAACGCGAAAGCGAAAACCGAU |
| (FTD) | | CAUGGCGGCAGCGGCAGCGGCAGCGGCAGCCUGCGC |
| | | GAACCGGGCCCGCCGGGCCUGAGCGGCAGCGGCAGC |
| | | GGCAGCGGCAGCACCCAUGUGCCGGGCGGCGGCAAC |
| | | AAAAAAAUUGGCAGCGGCAGCGGCAGCGGCAGCCAU |
| | | CAUAAACCGGGCGGCGGCCAGGUGGAAGUGAAAGGC |
| | | AGCGGCAGCGGCAGCGGCAGCACCCCGAACGUGCAG |
| | | AAAGAACAGGCGCAUAGCGAAGAACAUCUGGGCAGC |
| | | GGCAGCGGCAGCGGCAGCAGCAAAAUUGGCAGCACC |
| | | GAAAACCUGAAACAUCAGCCGGGCGGCGGCAAAGUG |
| | | GGCAGCGGCAGCGGCAGCGGCAGCAGCAAAUGCGGC |
| | | AGCAAAGAUAACAUUAAACAUGUGCCGGGCGGCGGC |
| | | AGCGUGGGCAGCGGCAGCGGCAGCGGCAGCACCGAA |
| | | AUUCCGGCGAGCGAACCGGAUGGCCCGAGCGUGGGC |
| | | CGCGCGAAAGGCCAGGAUGCGCCGGGCAGCGGCAGC |
| | | GGCAGCGGCAGCCGCGUGACCGAAGAUGAAAACGAU |
| | | GAACCGAUUGAAAUUCCGAGCGAAGAUGAUGGCACC |
| | | GUGGGCAGCGGCAGCGGCAGCGGCAGCCGCUAUCGC |
| | | AACCCGGUGAGCCAGUGCAUGCGCGGCGUGGGCAGC |
| | | GGCAGCGGCAGCGGCAGCCAUGCGCCGGAUGCGGGC |
| | | UGGGGCAACGGCAGCGGCAGCGGCAGCGGCAGCACC |
| | | ACCGAACAGGAUCUGAAAGAAUAUUUUAGCGGCAGC |
| | | GGCAGCGGCAGCGGCAGCGAAACCCAGGUGAAAGUG |
| | | AUGAGCCAGCGCCAUAUGAUUGAUGGCGGCAGCGGC |
| | | AGCGGCAGCGGCAGCCCGAACAGCAAACAGAGCCAG |
| | | GAUGAACCGCUGCGCAGCGGCAGCGGCAGCGGCAGC |
| | | GGCAGCGAUAUGACCGAAGAUGAACUGCGCGAAUUU |
| | | UUUAGCGGCAGCGGCAGCGGCAGCGGCAGCGGCGAA |
| | | GAUCUGAUUAUUAAAGGCGGCAGCGGCAGCGGCAGC |
| | | GGCAGCGCGGAACCGAAACAUAACAGCAACCGCCAG |
| | | CUGGAACGCAGCGGCCGCUUUGGCGGCAACCCGGGC |
| | | GGCUUUGGCAGCGGCAGCGGCAGCGGCAGCGGCUUU |
| | | AACGGCGGCUUUGGCAGCAGCAUGGAUAGCAAAAGC |
| | | AGCGGCGGCAGCGGCAGCGGCAGCGGCAGCCUGGGC |
| | | GAAAACGUGACCAUUGAAAGCGUGGCGGAUUAUUU |
| | | UAAACAGAUUGGCAGCGGCAGCGGCAGCGGCAGCAU |
| | | UAUUAAAACCAACAAAAAAACCGGCCAGGGCAGCGG |
| | | CAGCGGCAGCGGCAGCCGCGAAACCGGCAAACUGAA |
| | | AGGCAGCGGCAGCGGCAGCGGCAGCUUUGAUGGCAA |
| | | AGAAUUUAGCGGC |
| Lewy Body | α-synuclein, | AAAGGCCUGAGCAAAGGCAGCGGCAGCGGCAGCGGC |
| Dementia | Tau | AGCGAAAAAACCAAACAGGGCGUGGCGGAAGCGGCG |
| (LBD) | | GGCAAAACCAAAGAAGGCGUGGGCAGCGGCAGCGGC |
| | | AGCGGCAGCGUGGGCAGCAAAACCAAAGAAGGCGUG |
| | | GUGGGCAGCGGCAGCGGCAGCGGCAGCGCGGAAAAA |
| | | ACCAAAGAACAGGUGACCAACGUGGGCGGCGCGGUG |
| | | GUGACCGGCGGCAGCGGCAGCGGCAGCGGCAGCACC |
| | | GCGGUGGCGCAGAAAACCGUGGAAGGCAGCGGCAGC |
| | | GGCAGCGGCAGCUUUGUGAAAAAAGAUCAGCUGGGC |
| | | GGCAGCGGCAGCGGCAGCGGCAGCAUGGCGGAACCG |
| | | CGCCAGGAAUUUGAAGGCAGCGGCAGCGGCAGCGGC |
| | | AGCAACGCGAAAGCGAAAACCGAUCAUGGCGGCAGC |
| | | GGCAGCGGCAGCGGCAGCCUGCGCGAACCGGGCCCG |
| | | CCGGGCCUGAGCGGCAGCGGCAGCGGCAGCGGCAGC |
| | | ACCCAUGUGCCGGGCGGCGGCAACAAAAAAAUUGGC |
| | | AGCGGCAGCGGCAGCGGCAGCCAUCAUAAACCGGGC |
| | | GGCGGCCAGGUGGAAGUGAAAGGCAGCGGCAGCGGC |
| | | AGCGGCAGCACCCCGAACGUGCAGAAAGAACAGGCG |
| | | CAUAGCGAAGAACAUCUGGGCAGCGGCAGCGGCAGC |
| | | GGCAGCAGCAAAAUUGGCAGCACCGAAAACCUGAAA |
| | | CAUCAGCCGGGCGGCGGCAAAGUGGGCAGCGGCAGC |
| | | GGCAGCGGCAGCAGCAAAUGCGGCAGCAAAGAUAAC |
| | | AUUAAACAUGUGCCGGGCGGCGGCAGCGUGGGCAGC |
| | | GGCAGCGGCAGCGGCAGCACCGAAAUUCCGGCGAGC |
| | | GAACCGGAUGGCCCGAGCGUGGGCCGCGCGAAAGGC |
| | | CAGGAUGCGCCG |
| Multiple | α-synuclein | AAAGGCCUGAGCAAAGGCAGCGGCAGCGGCAGCGGC |
| System | | AGCGAAAAAACCAAACAGGGCGUGGCGGAAGCGGCG |
| Atrophy | | GGCAAAACCAAAGAAGGCGUGGGCAGCGGCAGCGGC |
| (MSA) | | AGCGGCAGCGUGGGCAGCAAAACCAAAGAAGGCGUG |
| | | GUGGGCAGCGGCAGCGGCAGCGGCAGCGCGGAAAAA |
| | | ACCAAAGAACAGGUGACCAACGUGGGCGGCGCGGUG |
| | | GUGACCGGCGGCAGCGGCAGCGGCAGCGGCAGCACC |
| | | GCGGUGGCGCAGAAAACCGUGGAAGGCAGCGGCAGC |
| | | GGCAGCGGCAGCUUUGUGAAAAAAGAUCAGCUGGGC |
| Parkinson′s | α-synuclein, | AAAGGCCUGAGCAAAGGCAGCGGCAGCGGCAGCGGC |
| Disease | Tau | AGCGAAAAAACCAAACAGGGCGUGGCGGAAGCGGCG |
| Dementia | | GGCAAAACCAAAGAAGGCGUGGGCAGCGGCAGCGGC |
| (PDD) | | AGCGGCAGCGUGGGCAGCAAAACCAAAGAAGGCGUG |
| | | GUGGGCAGCGGCAGCGGCAGCGGCAGCGCGGAAAAA |
| | | ACCAAAGAACAGGUGACCAACGUGGGCGGCGCGGUG |
| | | GUGACCGGCGGCAGCGGCAGCGGCAGCGGCAGCACC |
| | | GCGGUGGCGCAGAAAACCGUGGAAGGCAGCGGCAGC |
| | | GGCAGCGGCAGCUUUGUGAAAAAAGAUCAGCUGGGC |
| | | GGCAGCGGCAGCGGCAGCGGCAGCAUGGCGGAACCG |
| | | CGCCAGGAAUUUGAAGGCAGCGGCAGCGGCAGCGGC |
| | | AGCAACGCGAAAGCGAAAACCGAUCAUGGCGGCAGC |
| | | GGCAGCGGCAGCGGCAGCCUGCGCGAACCGGGCCCG |
| | | CCGGGCCUGAGCGGCAGCGGCAGCGGCAGCGGCAGC |
| | | ACCCAUGUGCCGGGCGGCGGCAACAAAAAAAUUGGC |
| | | AGCGGCAGCGGCAGCGGCAGCCAUCAUAAACCGGGC |
| | | GGCGGCCAGGUGGAAGUGAAAGGCAGCGGCAGCGGC |
| | | AGCGGCAGCACCCCGAACGUGCAGAAAGAACAGGCG |
| | | CAUAGCGAAGAACAUCUGGGCAGCGGCAGCGGCAGC |
| | | GGCAGCAGCAAAAUUGGCAGCACCGAAAACCUGAAA |
| | | CAUCAGCCGGGCGGCGGCAAAGUGGGCAGCGGCAGC |
| | | GGCAGCGGCAGCAGCAAAUGCGGCAGCAAAGAUAAC |
| | | AUUAAACAUGUGCCGGGCGGCGGCAGCGUGGGCAGC |
| | | GGCAGCGGCAGCGGCAGCACCGAAAUUCCGGCGAGC |
| | | GAACCGGAUGGCCCGAGCGUGGGCCGCGCGAAAGGC |
| | | CAGGAUGCGCCG |
| Progressive | Tau | AUGGCGGAACCGCGCCAGGAAUUUGAAGGCAGCGGC |
| Supranuclear | | AGCGGCAGCGGCAGCAACGCGAAAGCGAAAACCGAU |
| Palsy (PSP) | | CAUGGCGGCAGCGGCAGCGGCAGCGGCAGCCUGCGC |
| | | GAACCGGGCCCGCCGGGCCUGAGCGGCAGCGGCAGC |
| | | GGCAGCGGCAGCACCCAUGUGCCGGGCGGCGGCAAC |
| | | AAAAAAAUUGGCAGCGGCAGCGGCAGCGGCAGCCAU |
| | | CAUAAACCGGGCGGCGGCCAGGUGGAAGUGAAAGGC |
| | | AGCGGCAGCGGCAGCGGCAGCACCCCGAACGUGCAG |
| | | AAAGAACAGGCGCAUAGCGAAGAACAUCUGGGCAGC |
| | | GGCAGCGGCAGCGGCAGCAGCAAAAUUGGCAGCACC |
| | | GAAAACCUGAAACAUCAGCCGGGCGGCGGCAAAGUG |
| | | GGCAGCGGCAGCGGCAGCGGCAGCAGCAAAUGCGGC |
| | | AGCAAAGAUAACAUUAAACAUGUGCCGGGCGGCGGC |
| | | AGCGUGGGCAGCGGCAGCGGCAGCGGCAGCACCGAA |
| | | AUUCCGGCGAGCGAACCGGAUGGCCCGAGCGUGGGC |
| | | CGCGCGAAAGGCCAGGAUGCGCCG |

While each disease presents a separate profile of aggregation, it is not known if the aggregating proteins take part in other diseases or whether they talk to each other; therefore, one option to create a universal vaccine is to combine the open reading frames of all proteins identified above in Table 3.

1. A coding mRNA vaccine for a vaccine comprising
a) at least one 5′-cap structure
b) at least one heterologous 5′ untranslated region (5′-UTR)
c) at least one heterologous 3′ untranslated region (3′-UTR)
d) at least one signal peptide
e) at least one poly(A) tail
e) at least one coding open reading frame sequence to express epitopes of proteins comprising amyloid beta 42 (Aβ42), α-synuclein, tau protein, FUS protein, and TDP-43 protein or a combination thereof, linked operably to the 3′-UTR and the 5′-UTR.

2. The coding mRNA vaccine of claim 1, wherein the neurodegenerative disorders comprise Alzheimer's Disease (AD), Amyotrophic Lateral Sclerosis (ALS), Corticobasal Degeneration (CBD), Frontotemporal Dementia (FTD), Lewy Body Dementia (LBD), Multiple System Atrophy (MSA), Parkinson's Disease Dementia (PSD), and Progressive Supranuclear Palsy (PSP), or a combination thereof.

3. The coding mRNA vaccine of claim 1, wherein the 5′-cap comprises m7,3′-GpppG, m7G(5′)ppp(5′)(m7G), m7GpppN, m7GpppNm, m7GpppNmNm, m7GpppNmNmN, m7GpppN, m7G+m3′-5′-ppp-5′-Am, or m7GpppG.

4. The coding mRNA vaccine of claim 2, wherein the cap is m7G+m3′-5′-ppp-5′-Am.

5. The coding mRNA vaccine of claim 1, wherein the mRNA comprises a poly(A) sequence, preferably comprising 30 to 150 adenosine nucleotides.

6. The coding mRNA vaccine of claim 1, wherein the heterologous signal peptide is selected from a group of heterologous signal peptides comprising TTCGTGTTCCTGGTGCTGCTGCCTCTGGTGTCCAGCCAGTGTGTG, ATGTTCGTGTTCCTGGTGCTGCTGCCTCTGGTGTCCAGCCAGTGTGTG, MAKNLILALILAFVLTATVA, MALLWSVLLGLLQLGAVAPR, MKFLVNVALVFMVVYISYIYA, MKKNLILAVLALVSSILAN, MKKQSTIALALLPLLFTPVTKA, MKKTAIAIAVALAGFATVAQA, MKKTSFFLILVLLFTGCSG, MKLLTALALVVTMVASV, MKNWILVVLAVLAAVSSLAHA, MKWVTFISLLFLFSSAYS, MKYLLPTAAAGLLLLAAQPAMA, MLLSVPLLLGLLGLAVA, and MRRLLLTGAAALGCTAA, or a combination thereof.

7. The coding mRNA vaccine of claim 1, wherein the epitopes of α-synuclein comprise KGLSK, EKTKQGVAEAAGKTKEGV, VGSKTKEGVV, AEKTKEQVTNVGGAVVTG, TAVAQKTVE, FVKKDQLG, and FVKKDQLG, or a combination thereof.

8. The coding mRNA vaccine of claim 1, wherein the epitopes of amyloid beta 42 comprise EFRHDSGYEVHH, and EDVGSNKG, or a combination thereof.

9. The coding mRNA vaccine claim 1, wherein the epitopes of tau protein comprise MAEPRQEFE, NAKAKTDHG, LREPGPPGLS, THVPGGGNKKI, HHKPGGGQVEVK, TPNVQKEQAHSEEHL, SKIGSTENLKHQPGGGKV, SKCGSKDNIKHVPGGGSV, and TEIPASEPDGPSVGRAKGQDAP, or a combination thereof.

10. The coding mRNA vaccine of claim 1, wherein the epitopes of FUS proteins comprise LGENVTIESVADYFKQI, IIKTNKKTGQ, RETGKLK, and FDGKEFSG, or a combination thereof.

11. The coding mRNA vaccine of claim 1, wherein the epitopes of TDP-43 protein comprise RVTEDENDEPIEIPSEDDGTV, RYRNPVSQCMRGV, HAPDAGWGN, TTEQDLKEYFS, ETQVKVMSQRHMIDG, PNSKQSQDEPLRS, DMTEDELREFFS, GEDLIIKG, AEPKHNSNRQLERSGREGGNPGGF, and GFNGGFGSSMDSKSSG, or a combination thereof.

12. The coding mRNA vaccine of claims 7-11, wherein the epitopes are combined by using a linker comprising AAAGY (Alanine-Alanine-Alanine-Glycine-Tyrosine), AAY (alanine and tyrosine), APAAP (Alanine-Proline-Alanine-Alanine-Proline), EAAAK (Glutamic Acid-Alanine Linker), EFGGG (Glutamic Acid-Phenylalanine-Glycine-Glycine-Glycine), GGAGG (A slight variation of the GGGGS linker with an alanine residue), GGGGS (Glycine-Serine Linker), GGGS (linker is one of the simplest and commonly used linkers), GGGSGGG (linker consists of a longer sequence of glycine (G) and serine (S) residues), GGGSGGGGSGGG (linker with multiple glycine and serine residues), GGPGG (Glycine-Glycine-Proline-Glycine-Glycine), GGSGG: (An inversion of the standard GGGGS linker), GGSSG (Glycine-Glycine-Serine-Serine-Glycine), GGTGG (Glycine-Glycine-Threonine-Glycine-Glycine), GPGP (Glycine-Proline-Glycine-Proline), GPGPG (Glycine-Proline-Glycine-Proline-Glycine), GPGS (Glycine-Proline-Glycine-Serine), GSGPG (Glycine-Serine-Glycine-Proline-Glycine), GSSG (Glycine-Serine-Serine-Glycine), GSSGG (Glycine-Serine-Serine-Glycine-Glycine), GSTSG (Glycine-Serine-Threonine Linker), KK (Lysine-Lysine), KKKGS (Lysine-Glycine-Serine Linker), KLPGWSG: (A specific sequence), LEGGGS (Leucine-Glutamic Acid-Glycine-Glycine-Serine), NPGP (Asparagine-Proline-Glycine-Proline), SGGGG: (A variant of the GGGGS linker), SGSGS (Serine-Glycine-Serine-Glycine-Serine), SSGGG (Serine-Serine-Glycine-Glycine-Glycine), SSGSS (Serine-Serine-Glycine-Serine-Serine), TGGGS (Threonine-Glycine-Glycine-Glycine-Serine), TPGTG (Threonine-Proline-Glycine-Threonine-Glycine), TPP (Proline-Proline-Threonine), TPTPPT (Threonine-Proline-Threonine-Proline-Proline-Threonine), TSGSG: (A variant of the GSTSG linker), , TSGTSG (Threonine-Serine-Glycine-Threonine-Serine-Glycine), and XTEN: (A synthetic, non-immunogenic linker), or a combination thereof.

13. The coding mRNA vaccine of claim 1, wherein the Open Reading Frame to treat AD comprises
GAAUUUCGCCAUGAUAGCGGCUAUGAAGUGCAUCAUGGCAGCGGCAGCGG
CAGCGGCAGCGAAGAUGUGGGCAGCAACAAAGGCGGCAGCGGCAGCGGCA
GCGGCAGCAUGGCGGAACCGCGCCAGGAAUUUGAAGGCAGCGGCAGCGGC
AGCGGCAGCAACGCGAAAGCGAAAACCGAUCAUGGCGGCAGCGGCAGCGG
CAGCGGCAGCCUGCGCGAACCGGGCCCGCCGGGCCUGAGCGGCAGCGGCA
GCGGCAGCGGCAGCACCCAUGUGCCGGGCGGCGGCAACAAAAAAAUUGGC
AGCGGCAGCGGCAGCGGCAGCCAUCAUAAACCGGGCGGCGGCCAGGUGGA
AGUGAAAGGCAGCGGCAGCGGCAGCGGCAGCACCCCGAACGUGCAGAAAG
AACAGGCGCAUAGCGAAGAACAUCUGGGCAGCGGCAGCGGCAGCGGCAGC
AGCAAAAUUGGCAGCACCGAAAACCUGAAACAUCAGCCGGGCGGCGGCAA
AGUGGGCAGCGGCAGCGGCAGCGGCAGCAGCAAAUGCGGCAGCAAAGAUA
ACAUUAAACAUGUGCCGGGCGGCGGCAGCGUGGGCAGCGGCAGCGGCAGC
GGCAGCACCGAAAUUCCGGCGAGCGAACCGGAUGGCCCGAGCGUGGGCCG
CGCGAAAGGCCAGGAUGCGCCG.

14. The coding mRNA vaccine of claim 1, wherein the open reading frame to prevent or treat ALS comprises
CUGGGCGAAAACGUGACCAUUGAAAGCGUGGCGGAUUAUUUUAAACAGA
UUGGCAGCGGCAGCGGCAGCGGCAGCAUUAUUAAAACCAACAAAAAAACC
GGCCAGGGCAGCGGCAGCGGCAGCGGCAGCCGCGAAACCGGCAAACUGAA
AGGCAGCGGCAGCGGCAGCGGCAGCUUUGAUGGCAAAGAAUUUAGCGGCG
GCAGCGGCAGCGGCAGCGGCAGCCGCGUGACCGAAGAUGAAAACGAUGAA
CCGAUUGAAAUUCCGAGCGAAGAUGAUGGCACCGUGGGCAGCGGCAGCGG
CAGCGGCAGCCGCUAUCGCAACCCGGUGAGCCAGUGCAUGCGCGGCGUGG
GCAGCGGCAGCGGCAGCGGCAGCCAUGCGCCGGAUGCGGGCUGGGGCAAC
GGCAGCGGCAGCGGCAGCGGCAGCACCACCGAACAGGAUCUGAAAGAAUA
UUUUAGCGGCAGCGGCAGCGGCAGCGGCAGCGAAACCCAGGUGAAAGUGA
UGAGCCAGCGCCAUAUGAUUGAUGGCGGCAGCGGCAGCGGCAGCGGCAGC
CCGAACAGCAAACAGAGCCAGGAUGAACCGCUGCGCAGCGGCAGCGGCAG
CGGCAGCGGCAGCGAUAUGACCGAAGAUGAACUGCGCGAAUUUUUUAGCG
GCAGCGGCAGCGGCAGCGGCAGCGGCGAAGAUCUGAUUAUUAAAGGCGGC
AGCGGCAGCGGCAGCGGCAGCGCGGAACCGAAACAUAACAGCAACCGCCA
GCUGGAACGCAGCGGCCGCUUUGGCGGCAACCCGGGCGGCUUUGGCAGCG
GCAGCGGCAGCGGCAGCGGCUUUAACGGCGGCUUUGGCAGCAGCAUGGAU
AGCAAAAGCAGCGGC.

15. The coding mRNA vaccine of claim 1, wherein the open reading frame to prevent or treat CBD and PSP comprises
AUGGCGGAACCGCGCCAGGAAUUUGAAGGCAGCGGCAGCGGCAGCGGCAG
CAACGCGAAAGCGAAAACCGAUCAUGGCGGCAGCGGCAGCGGCAGCGGCA
GCCUGCGCGAACCGGGCCCGCCGGGCCUGAGCGGCAGCGGCAGCGGCAGC
GGCAGCACCCAUGUGCCGGGCGGCGGCAACAAAAAAAUUGGCAGCGGCAG
CGGCAGCGGCAGCCAUCAUAAACCGGGCGGCGGCCAGGUGGAAGUGAAAG
GCAGCGGCAGCGGCAGCGGCAGCACCCCGAACGUGCAGAAAGAACAGGCG
CAUAGCGAAGAACAUCUGGGCAGCGGCAGCGGCAGCGGCAGCAGCAAAAU
UGGCAGCACCGAAAACCUGAAACAUCAGCCGGGCGGCGGCAAAGUGGGCA
GCGGCAGCGGCAGCGGCAGCAGCAAAUGCGGCAGCAAAGAUAACAUUAAA
CAUGUGCCGGGCGGCGGCAGCGUGGGCAGCGGCAGCGGCAGCGGCAGCAC
CGAAAUUCCGGCGAGCGAACCGGAUGGCCCGAGCGUGGGCCGCGCGAAAG
GCCAGGAUGCGCCG.

16. The coding mRNA vaccine of claim 1, wherein the open reading frame to prevent or treat FTD comprises
AUGGCGGAACCGCGCCAGGAAUUUGAAGGCAGCGGCAGCGGCAGCGGCAG
CAACGCGAAAGCGAAAACCGAUCAUGGCGGCAGCGGCAGCGGCAGCGGCA
GCCUGCGCGAACCGGGCCCGCCGGGCCUGAGCGGCAGCGGCAGCGGCAGC
GGCAGCACCCAUGUGCCGGGCGGCGGCAACAAAAAAAUUGGCAGCGGCAG
CGGCAGCGGCAGCCAUCAUAAACCGGGCGGCGGCCAGGUGGAAGUGAAAG
GCAGCGGCAGCGGCAGCGGCAGCACCCCGAACGUGCAGAAAGAACAGGCG
CAUAGCGAAGAACAUCUGGGCAGCGGCAGCGGCAGCGGCAGCAGCAAAAU
UGGCAGCACCGAAAACCUGAAACAUCAGCCGGGCGGCGGCAAAGUGGGCA
GCGGCAGCGGCAGCGGCAGCAGCAAAUGCGGCAGCAAAGAUAACAUUAAA
CAUGUGCCGGGCGGCGGCAGCGUGGGCAGCGGCAGCGGCAGCGGCAGCAC
CGAAAUUCCGGCGAGCGAACCGGAUGGCCCGAGCGUGGGCCGCGCGAAAG
GCCAGGAUGCGCCGGGCAGCGGCAGCGGCAGCGGCAGCCGCGUGACCGAA
GAUGAAAACGAUGAACCGAUUGAAAUUCCGAGCGAAGAUGAUGGCACCGU
GGGCAGCGGCAGCGGCAGCGGCAGCCGCUAUCGCAACCCGGUGAGCCAGU
GCAUGCGCGGCGUGGGCAGCGGCAGCGGCAGCGGCAGCCAUGCGCCGGAU
GCGGGCUGGGGCAACGGCAGCGGCAGCGGCAGCGGCAGCACCACCGAACA
GGAUCUGAAAGAAUAUUUUAGCGGCAGCGGCAGCGGCAGCGGCAGCGAAA
CCCAGGUGAAAGUGAUGAGCCAGCGCCAUAUGAUUGAUGGCGGCAGCGGC
AGCGGCAGCGGCAGCCCGAACAGCAAACAGAGCCAGGAUGAACCGCUGCG
CAGCGGCAGCGGCAGCGGCAGCGGCAGCGAUAUGACCGAAGAUGAACUGC
GCGAAUUUUUUAGCGGCAGCGGCAGCGGCAGCGGCAGCGGCGAAGAUCUG
AUUAUUAAAGGCGGCAGCGGCAGCGGCAGCGGCAGCGCGGAACCGAAACA
UAACAGCAACCGCCAGCUGGAACGCAGCGGCCGCUUUGGCGGCAACCCGG
GCGGCUUUGGCAGCGGCAGCGGCAGCGGCAGCGGCUUUAACGGCGGCUUU
GGCAGCAGCAUGGAUAGCAAAAGCAGCGGCGGCAGCGGCAGCGGCAGCGG
CAGCCUGGGCGAAAACGUGACCAUUGAAAGCGUGGCGGAUUAUUUUAAAC
AGAUUGGCAGCGGCAGCGGCAGCGGCAGCAUUAUUAAAACCAACAAAAAA
ACCGGCCAGGGCAGCGGCAGCGGCAGCGGCAGCCGCGAAACCGGCAAACU
GAAAGGCAGCGGCAGCGGCAGCGGCAGCUUUGAUGGCAAAGAAUUUAGCG
GC.

17. The coding mRNA vaccine of claim 1, wherein the open reading frame to prevent or treat LBD and PDD comprises
AAAGGCCUGAGCAAAGGCAGCGGCAGCGGCAGCGGCAGCGAAAAAACCAA
ACAGGGCGUGGCGGAAGCGGCGGGCAAAACCAAAGAAGGCGUGGGCAGCG
GCAGCGGCAGCGGCAGCGUGGGCAGCAAAACCAAAGAAGGCGUGGUGGGC
AGCGGCAGCGGCAGCGGCAGCGCGGAAAAAACCAAAGAACAGGUGACCAA
CGUGGGCGGCGCGGUGGUGACCGGCGGCAGCGGCAGCGGCAGCGGCAGCA
CCGCGGUGGCGCAGAAAACCGUGGAAGGCAGCGGCAGCGGCAGCGGCAGC
UUUGUGAAAAAAGAUCAGCUGGGCGGCAGCGGCAGCGGCAGCGGCAGCAU
GGCGGAACCGCGCCAGGAAUUUGAAGGCAGCGGCAGCGGCAGCGGCAGCA
ACGCGAAAGCGAAAACCGAUCAUGGCGGCAGCGGCAGCGGCAGCGGCAGC
CUGCGCGAACCGGGCCCGCCGGGCCUGAGCGGCAGCGGCAGCGGCAGCGG
CAGCACCCAUGUGCCGGGCGGCGGCAACAAAAAAAUUGGCAGCGGCAGCG
GCAGCGGCAGCCAUCAUAAACCGGGCGGCGGCCAGGUGGAAGUGAAAGGC
AGCGGCAGCGGCAGCGGCAGCACCCCGAACGUGCAGAAAGAACAGGCGCA
UAGCGAAGAACAUCUGGGCAGCGGCAGCGGCAGCGGCAGCAGCAAAAUUG
GCAGCACCGAAAACCUGAAACAUCAGCCGGGCGGCGGCAAAGUGGGCAGC
GGCAGCGGCAGCGGCAGCAGCAAAUGCGGCAGCAAAGAUAACAUUAAACA
UGUGCCGGGCGGCGGCAGCGUGGGCAGCGGCAGCGGCAGCGGCAGCACCG
AAAUUCCGGCGAGCGAACCGGAUGGCCCGAGCGUGGGCCGCGCGAAAGGC
CAGGAUGCGCCG.

18. The coding mRNA vaccine of claim 1, wherein the open reading frame to prevent or treat MSA comprises
AAAGGCCUGAGCAAAGGCAGCGGCAGCGGCAGCGGCAGCGAAAAAACCAA
ACAGGGCGUGGCGGAAGCGGCGGGCAAAACCAAAGAAGGCGUGGGCAGCG
GCAGCGGCAGCGGCAGCGUGGGCAGCAAAACCAAAGAAGGCGUGGUGGGC
AGCGGCAGCGGCAGCGGCAGCGCGGAAAAAACCAAAGAACAGGUGACCAA
CGUGGGCGGCGCGGUGGUGACCGGCGGCAGCGGCAGCGGCAGCGGCAGCA
CCGCGGUGGCGCAGAAAACCGUGGAAGGCAGCGGCAGCGGCAGCGGCAGC
UUUGUGAAAAAAGAUCAGCUGGGC.

19. The coding mRNA vaccine of claim 1, where in the open reading frames are combined into a single open reading frame to create a universal vaccine against the dementia-causing neurodegenerative disorders.

20. The coding mRNA vaccine of claim 1, wherein RNA sequences are codon-optimized by replacing uridine (U) either wholly or partially by pseudouridine and N1-methyl pseudouridine pseudouridine (Y), or a combination thereof.

21. The coding mRNA vaccine of claim 1, wherein the mRNA molecule additionally contains a functional group capable of enhancing the immune response of the mRNA molecule.

22. The coding mRNA vaccine of claim 1, wherein the coding mRNA is a self-replicating RNA, a circular RNA, or a replicon RNA.

23. The coding mRNA vaccine of claim 1, wherein the vaccine optionally comprises at least one pharmaceutically acceptable carrier.

24. The coding mRNA vaccine of claim 1, wherein the coding mRNA is complexed or associated with or at least partially complexed or partially associated with one or more cationic or polycationic compounds, preferably cationic or polycationic polymer, cationic or polycationic polysaccharide, cationic or polycationic lipid, cationic or polycationic protein, cationic or polycationic peptide, or any combinations thereof.

25. The coding mRNA vaccine of claim 24, wherein the coding mRNA is complexed or associated with one or more lipids, thereby forming liposomes, lipid nanoparticles (LNP), lipoplexes, and nanoliposomes.

26. The coding mRNA vaccine of claim 25, wherein the LNP essentially consists of
(i) at least one cationic lipid;
(ii) at least one neutral lipid;
(iii) at least one steroid or steroid analog; and
(iv) at least once a PEG-lipid, Wherein (i) to (iv) are in a molar ratio of about 20-60% cationic lipid, 5-25% neutral lipid, 25-55% sterol, and ½-15% PEG-lipid.

27. The coding mRNA vaccine of claim 25, where the LNP formulation comprises the steps of mixing D-Lin-MC3-DMA, DSPC, cholesterol, and DMG-PEG 2000 in an absolute ethanol solution, adding the mixture into a citrate buffer solution, and extruding the mixture by a liposome extruder to obtain the liposome nanoparticle.

28. The vaccine of claim 25, wherein the LNP is lyophilized.

29. The coding mRNA vaccine of claim 1, wherein the vaccine elicits an adaptive immune response.

30. A method of treating or preventing a neurodegenerative disorder, wherein the method comprises applying or administering to a human subject needing a coding mRNA vaccine as defined in any of the above claims.

31. The method of claim 30, wherein the disorder comprises Alzheimer's Disease, Amyotrophic lateral sclerosis, Chronic Traumatic Encephalopathy, Corticobasal Degeneration, Lewy Bodies Dementia, Frontotemporal Dementia, Huntington's disease, Multiple sclerosis, Multiple System Atrophy, Parkinson's Disease, Prion disease, Progressive Supranuclear Palsy, and Tauopathies, or a combination thereof.

32. The method of claim 31, wherein the vaccines to prevent or treat the listed disorders are combined as a single vaccine to serve as a universal vaccine against dementia-causing neurodegenerative disorders.


SEQUENCE LISTING (42 sequences)

SEQ ID NO: 1 - RNA, 42 nt, synthetic construct
  source 1..42: mol_type = other RNA
gaaaaacagaccggccccacagaccagagagaacccgccacc

SEQ ID NO: 2 - RNA, 31 nt, unidentified
  source 1..31: mol_type = mRNA
agcggccgggcgcgcccgggccagccagggg

SEQ ID NO: 3 - RNA, 672 nt, Synthetic construct
  source 1..672: mol_type = other RNA
gaatttcgccatgatagcggctatgaagtgcatcatggcagcggcagcggcagcggcagcgaagatgtgggcagcaacaaaggcggcagcggcagcggcagcggcagcatggcggaaccgcgccaggaatttgaaggcagcggcagcggcagcggcagcaacgcgaaagcgaaaaccgatcatggcggcagcggcagcggcagcggcagcctgcgcgaaccgggcccgccgggcctgagcggcagcggcagcggcagcggcagcacccatgtgccgggcggcggcaacaaaaaaattggcagcggcagcggcagcggcagccatcataaaccgggcggcggccaggtggaagtgaaaggcagcggcagcggcagcggcagcaccccgaacgtgcagaaagaacaggcgcatagcgaagaacatctgggcagcggcagcggcagcggcagcagcaaaattggcagcaccgaaaacctgaaacatcagccgggcggcggcaaagtgggcagcggcagcggcagcggcagcagcaaatgcggcagcaaagataacattaaacatgtgccgggcggcggcagcgtgggcagcggcagcggcagcggcagcaccgaaattccggcgagcgaaccggatggcccgagcgtgggccgcgcgaaaggccaggatgcgccg

SEQ ID NO: 4 - RNA, 864 nt, Synthetic construct
  source 1..864: mol_type = other RNA
ctgggcgaaaacgtgaccattgaaagcgtggcggattattttaaacagattggcagcggcagcggcagcggcagcattattaaaaccaacaaaaaaaccggccagggcagcggcagcggcagcggcagccgcgaaaccggcaaactgaaaggcagcggcagcggcagcggcagctttgatggcaaagaatttagcggcggcagcggcagcggcagcggcagccgcgtgaccgaagatgaaaacgatgaaccgattgaaattccgagcgaagatgatggcaccgtgggcagcggcagcggcagcggcagccgctatcgcaacccggtgagccagtgcatgcgcggcgtgggcagcggcagcggcagcggcagccatgcgccggatgcgggctggggcaacggcagcggcagcggcagcggcagcaccaccgaacaggatctgaaagaatattttagcggcagcggcagcggcagcggcagcgaaacccaggtgaaagtgatgagccagcgccatatgattgatggcggcagcggcagcggcagcggcagcccgaacagcaaacagagccaggatgaaccgctgcgcagcggcagcggcagcggcagcggcagcgatatgaccgaagatgaactgcgcgaattttttagcggcagcggcagcggcagcggcagcggcgaagatctgattattaaaggcggcagcggcagcggcagcggcagcgcggaaccgaaacataacagcaaccgccagctggaacgcagcggccgctttggcggcaacccgggcggctttggcagcggcagcggcagcggcagcggctttaacggcggctttggcagcagcatggatagcaaaagcagcggc

SEQ ID NO: 5 - RNA, 564 nt, Synthetic construct
  source 1..564: mol_type = other RNA
atggcggaaccgcgccaggaatttgaaggcagcggcagcggcagcggcagcaacgcgaaagcgaaaaccgatcatggcggcagcggcagcggcagcggcagcctgcgcgaaccgggcccgccgggcctgagcggcagcggcagcggcagcggcagcacccatgtgccgggcggcggcaacaaaaaaattggcagcggcagcggcagcggcagccatcataaaccgggcggcggccaggtggaagtgaaaggcagcggcagcggcagcggcagcaccccgaacgtgcagaaagaacaggcgcatagcgaagaacatctgggcagcggcagcggcagcggcagcagcaaaattggcagcaccgaaaacctgaaacatcagccgggcggcggcaaagtgggcagcggcagcggcagcggcagcagcaaatgcggcagcaaagataacattaaacatgtgccgggcggcggcagcgtgggcagcggcagcggcagcggcagcaccgaaattccggcgagcgaaccggatggcccgagcgtgggccgcgcgaaaggccaggatgcgccg

SEQ ID NO: 6 - RNA, 1452 nt, Synthetic construct
  source 1..1452: mol_type = other RNA
atggcggaaccgcgccaggaatttgaaggcagcggcagcggcagcggcagcaacgcgaaagcgaaaaccgatcatggcggcagcggcagcggcagcggcagcctgcgcgaaccgggcccgccgggcctgagcggcagcggcagcggcagcggcagcacccatgtgccgggcggcggcaacaaaaaaattggcagcggcagcggcagcggcagccatcataaaccgggcggcggccaggtggaagtgaaaggcagcggcagcggcagcggcagcaccccgaacgtgcagaaagaacaggcgcatagcgaagaacatctgggcagcggcagcggcagcggcagcagcaaaattggcagcaccgaaaacctgaaacatcagccgggcggcggcaaagtgggcagcggcagcggcagcggcagcagcaaatgcggcagcaaagataacattaaacatgtgccgggcggcggcagcgtgggcagcggcagcggcagcggcagcaccgaaattccggcgagcgaaccggatggcccgagcgtgggccgcgcgaaaggccaggatgcgccgggcagcggcagcggcagcggcagccgcgtgaccgaagatgaaaacgatgaaccgattgaaattccgagcgaagatgatggcaccgtgggcagcggcagcggcagcggcagccgctatcgcaacccggtgagccagtgcatgcgcggcgtgggcagcggcagcggcagcggcagccatgcgccggatgcgggctggggcaacggcagcggcagcggcagcggcagcaccaccgaacaggatctgaaagaatattttagcggcagcggcagcggcagcggcagcgaaacccaggtgaaagtgatgagccagcgccatatgattgatggcggcagcggcagcggcagcggcagcccgaacagcaaacagagccaggatgaaccgctgcgcagcggcagcggcagcggcagcggcagcgatatgaccgaagatgaactgcgcgaattttttagcggcagcggcagcggcagcggcagcggcgaagatctgattattaaaggcggcagcggcagcggcagcggcagcgcggaaccgaaacataacagcaaccgccagctggaacgcagcggccgctttggcggcaacccgggcggctttggcagcggcagcggcagcggcagcggctttaacggcggctttggcagcagcatggatagcaaaagcagcggcggcagcggcagcggcagcggcagcctgggcgaaaacgtgaccattgaaagcgtggcggattattttaaacagattggcagcggcagcggcagcggcagcattattaaaaccaacaaaaaaaccggccagggcagcggcagcggcagcggcagccgcgaaaccggcaaactgaaaggcagcggcagcggcagcggcagctttgatggcaaagaatttagcggc

SEQ ID NO: 7 - RNA, 912 nt, Synthetic construct
  source 1..912: mol_type = other RNA
aaaggcctgagcaaaggcagcggcagcggcagcggcagcgaaaaaaccaaacagggcgtggcggaagcggcgggcaaaaccaaagaaggcgtgggcagcggcagcggcagcggcagcgtgggcagcaaaaccaaagaaggcgtggtgggcagcggcagcggcagcggcagcgcggaaaaaaccaaagaacaggtgaccaacgtgggcggcgcggtggtgaccggcggcagcggcagcggcagcggcagcaccgcggtggcgcagaaaaccgtggaaggcagcggcagcggcagcggcagctttgtgaaaaaagatcagctgggcggcagcggcagcggcagcggcagcatggcggaaccgcgccaggaatttgaaggcagcggcagcggcagcggcagcaacgcgaaagcgaaaaccgatcatggcggcagcggcagcggcagcggcagcctgcgcgaaccgggcccgccgggcctgagcggcagcggcagcggcagcggcagcacccatgtgccgggcggcggcaacaaaaaaattggcagcggcagcggcagcggcagccatcataaaccgggcggcggccaggtggaagtgaaaggcagcggcagcggcagcggcagcaccccgaacgtgcagaaagaacaggcgcatagcgaagaacatctgggcagcggcagcggcagcggcagcagcaaaattggcagcaccgaaaacctgaaacatcagccgggcggcggcaaagtgggcagcggcagcggcagcggcagcagcaaatgcggcagcaaagataacattaaacatgtgccgggcggcggcagcgtgggcagcggcagcggcagcggcagcaccgaaattccggcgagcgaaccggatggcccgagcgtgggccgcgcgaaaggccaggatgcgccg

SEQ ID NO: 8 - RNA, 324 nt, Synthetic construct
  source 1..324: mol_type = other RNA
aaaggcctgagcaaaggcagcggcagcggcagcggcagcgaaaaaaccaaacagggcgtggcggaagcggcgggcaaaaccaaagaaggcgtgggcagcggcagcggcagcggcagcgtgggcagcaaaaccaaagaaggcgtggtgggcagcggcagcggcagcggcagcgcggaaaaaaccaaagaacaggtgaccaacgtgggcggcgcggtggtgaccggcggcagcggcagcggcagcggcagcaccgcggtggcgcagaaaaccgtggaaggcagcggcagcggcagcggcagctttgtgaaaaaagatcagctgggc

SEQ ID NO: 9 - RNA, 912 nt, Synthetic construct; identical to SEQ ID NO: 7
  source 1..912: mol_type = other RNA

SEQ ID NO: 10 - RNA, 564 nt, Synthetic construct; identical to SEQ ID NO: 5
  source 1..564: mol_type = other RNA

SEQ ID NO: 11 - protein, 12 aa, Synthetic construct
EFRHDSGYEVHH

SEQ ID NO: 12 - protein, 8 aa, Synthetic construct
EDVGSNKG

SEQ ID NO: 13 - protein, 9 aa, Synthetic construct
MAEPRQEFE

SEQ ID NO: 14 - protein, 9 aa, synthetic construct
NAKAKTDHG

SEQ ID NO: 15 - protein, 10 aa, Synthetic construct
LREPGPPGLS

SEQ ID NO: 16 - protein, 11 aa, Synthetic construct
THVPGGGNKKI

SEQ ID NO: 17 - protein, 12 aa, Synthetic construct
HHKPGGGQVEVK

SEQ ID NO: 18 - protein, 15 aa, Synthetic construct
TPNVQKEQAHSEEHL

SEQ ID NO: 19 - protein, 18 aa, Synthetic construct
SKIGSTENLKHQPGGGKV

SEQ ID NO: 20 - protein, 18 aa, Synthetic construct
SKCGSKDNIKHVPGGGSV

SEQ ID NO: 21 - protein, 22 aa, Synthetic construct
TEIPASEPDGPSVGRAKGQDAP

SEQ ID NO: 22 - protein, 5 aa, Synthetic construct
KGLSK

SEQ ID NO: 23 - protein, 18 aa, Synthetic construct
EKTKQGVAEAAGKTKEGV

SEQ ID NO: 24 - protein, 10 aa, Synthetic construct
VGSKTKEGVV

SEQ ID NO: 25 - protein, 18 aa, Synthetic construct
AEKTKEQVTNVGGAVVTG

SEQ ID NO: 26 - protein, 17 aa, Synthetic construct
TAVAQKTVEFVKKDQLG

SEQ ID NO: 27 - protein, 21 aa, Synthetic construct
RVTEDENDEPIEIPSEDDGTV

SEQ ID NO: 28 - protein, 13 aa, Synthetic construct
RYRNPVSQCMRGV

SEQ ID NO: 29 - protein, 9 aa, Synthetic construct
HAPDAGWGN

SEQ ID NO: 30 - protein, 11 aa, Synthetic construct
TTEQDLKEYFS

SEQ ID NO: 31 - protein, 15 aa, Synthetic construct
ETQVKVMSQRHMIDG

SEQ ID NO: 32 - protein, 13 aa, Synthetic construct
PNSKQSQDEPLRS

SEQ ID NO: 33 - protein, 12 aa, Synthetic construct
DMTEDELREFFS

SEQ ID NO: 34 - protein, 8 aa, Synthetic construct
GEDLIIKG

SEQ ID NO: 35 - protein, 24 aa, Synthetic construct
AEPKHNSNRQLERSGRFGGNPGGF

SEQ ID NO: 36 - protein, 16 aa, Synthetic construct
GFNGGFGSSMDSKSSG

SEQ ID NO: 37 - protein, 17 aa, Synthetic construct
LGENVTIESVADYFKQI

SEQ ID NO: 38 - protein, 10 aa, Synthetic construct
IIKTNKKTGQ

SEQ ID NO: 39 - protein, 7 aa, Synthetic construct
RETGKLK

SEQ ID NO: 40 - protein, 8 aa, Synthetic construct
FDGKEFSG

SEQ ID NO: 41 - RNA, 216 nt, unidentified
  source 1..216: mol_type = mRNA
gcagcgcccccccgccgggaccccgagccccccgacccgggcccaggagccccaccccaccgccccaccaccacccgcagccagacacccccaagcacgcagcaagcagccaaaacgcagccagccacacccccacgggaaacagcaggaaaccagcaaaaacgaaagaacaagcaacaaccccagggggcaacggccagccacacccggagcagc

SEQ ID NO: 42 - RNA, 107 nt, Synthetic construct
  source 1..107: mol_type = other RNA
aaaaaaaaaaaaaaaaaaaaaaaaaaaaaagcaagacaaaaaaaaaaaaaaaaaaaaaaaaaaaaaaaaaaaaaaaaaaaaaaaaaaaaaaaaaaaaaaaaaaaaaa